(19) United States
(12) Patent Application Publication
(10) Pub. No.: US 2026/0258444 A1
(43) Pub. Date: Sep. 3, 2026

(54) ITCH MUTANTS FOR THE TREATMENT OF MEMORY AND LEARNING DEFECTS

(71) Applicant: NATIONAL INSTITUTE OF IMMUNOLOGY, Delhi (IN)

(72) Inventors: Pushkar SHARMA, Delhi (IN); Monika CHAUHAN, Delhi (IN)

(21) Appl. No.: 18/878,559

(22) PCT Filed: Sep. 15, 2023
(86) PCT No.: PCT/IN2023/050862

§ 371 Date: Dec. 23, 2024

(30) Foreign Application Priority Data
Sep. 16, 2022 (IN) 202211052973

(51) Int. Cl. C12N 15/86; A61K 38/45; A61K 48/00; A61P 25/28; C12N 9/10

(52) U.S. Cl. CPC C12N 15/86; A61K 38/45; A61K 48/0033; A61K 48/005; A61P 25/28; C12N 9/104; C12N 2750/14143

(57) ABSTRACT

The present disclosure provides methods for treating a cognitive impairment due to Itch dysregulation in a patient by administering a viral vector comprising a polynucleotide sequence encoding an Itch mutant to the patient. The Itch mutant comprises a mutation selected from the group consisting of: T222A, S232A, K391R, and a combination thereof. The administration of the viral vector increases the levels of TAp73 and decreases the levels of proliferating cell nuclear antigen (PCNA) and cleaved caspase-3 in the patients. The methods of the present disclosure reduce the neurodegeneration and improve the cognitive and functional decline in AD patients.

### CROSS-REFERENCE TO RELATED APPLICATIONS

The present application is a filing under 35 U.S.C. 371 as the National Stage of International Application No. PCT/IN2023/050862, filed Sep. 15, 2023, which claims priority to Indian Application No. 202211052973 filed with the Intellectual Property Office of India on Sep. 16, 2022, both of which are incorporated herein by reference in their entirety for all purposes.

### FIELD OF THE DISCLOSURE

The present invention relates to methods for treating a mild cognitive impairment (MCI) in a patient. In particular, the present invention relates to the method and composition for treating a cognitive impairment due to Itch dysregulation by administering to the patient a viral vector comprising a polynucleotide sequence encoding an Itch mutant.

### INCORPORATION BY REFERENCE OF THE SEQUENCE LISTING

A Sequence Listing in XML format, submitted under 37 C.F.R. § 1.821, entitled “Seq_Listing_FP11718”, ~ 79 KB in size, generated on Dec. 16, 2024, and filed via EFS-Web, is provided in lieu of a paper copy. This Sequence Listing is hereby incorporated herein by reference into the specification for its disclosures.

### BACKGROUND OF THE DISCLOSURE

Neurological disorders are generally characterized by degeneration of the neurons. Cell cycle related neuronal apoptosis (CRNA) is a small yet common aspect of various neurodegenerative disorders such as cognitive impairment. Now a days the cognitive impairment associated with brain diseases remains a major cause of death throughout the developed world. The cell death in brain could be the direct cause of the neurodegenerative disease or a consequence of other processes that damage brain cells progressively. Due to the neuronal cell death, the communication between the neuronal cells is gradually destroyed thereby affecting the functioning of the brain. Neurodegenerative diseases can influence an individual's movement, speech, memory, intelligence, and other neural effects. The development of new therapies depends on the identification of suitable targets for drug activity. Decline in memory and learning is one of the major pathophysiology associated with the late stages of the disease. Recent advances in Alzheimer's disease (AD) research suggest that 65-70% of clinical trials for AD have evolved from the therapeutics directed against amyloid beta (AB) as a pharmacological target (Cummings 2018, Cummings, Lee et al. 2020). But most of these trials have failed to reach their respective end and are non-curative. This suggests that there is a wide translational gap between pre-clinical and clinical trials of AD (Mehta, Short et al. 2015). Furthermore, currently approved treatments for AD are more toward curing the symptoms of the disease and the agents that improve cognition. Therefore, there is a dire need of new approach for the therapeutic pipeline. E3 ligases play an important role in neurodegeneration. They are also emerging as drug able targets in various diseases including neurodegenerative disorders (Popovic, Vucic et al. 2014, Upadhyay, Joshi et al. 2017). These studies have also provided cues for some E3 regulators (like E6-AP, ITCH, MGRN1, and HACE1) in neurodevelopment and neurodegenerative disorders. Since cell-cycle related neuronal apoptosis (CRNA) contributes to neuronal loss in AD (Busser, Geldmacher et al. 1998, Yang, Mufson et al. 2003, Webber, Raina et al. 2005), the strategies preventing CRNA may be useful.

Itch has been shown to cause the degradation of TAp73 and thereby induce CRNA. The present invention uses viral vectors to express mutants of Itch to prevent degradation of TAp73 and thereby prevent CRNA. The present disclosure provides methods for treating a cognitive impairment in patients caused by Itch dysregulations by administering a viral vector expressing a mutant form of Itch.

### SUMMARY OF THE DISCLOSURE

The present disclosure provides a method for treating the cognitive impairment due to Itch dysregulation in a patient in need thereof, comprising administering to the patient a viral vector comprising a polynucleotide sequence encoding an Itch mutant.

The present disclosure provides a method for reducing neurodegeneration in a patient suffering from cognitive impairment or at a risk of developing AD, comprising administering to the patient a viral vector having a polynucleotide sequence encoding an Itch mutant.

The present disclosure provides a method for treating a mild cognitive impairment (MCI) in a patient, comprising administering to the patient a viral vector comprising a polynucleotide sequence encoding an Itch mutant, wherein the Itch mutant comprises a mutation selected from the group consisting of: T222A, S232A, K391R and a combination thereof.

The present disclosure also provides a method for treating a pre-clinical stage cognitive impairment in a patient, comprising administering to the patient a viral vector comprising a polynucleotide sequence encoding an Itch mutant.

The present disclosure provides a composition for use in treating a cognitive impairment due to Itch dysregulation, wherein the composition comprises a viral vector comprising a polynucleotide sequence encoding an Itch mutant. In some embodiments, the viral vector comprises a polynucleotide sequence that encodes an Itch mutant, wherein the Itch mutant comprises T222A, S232A, or K391R mutation or a combination thereof.

The present disclosure provides use of a viral vector comprising a polynucleotide sequence encoding an Itch mutant for treating various clinical stages of cognitive impairment due to Itch dysregulation as described herein.

The present disclosure provides a viral vector comprising a polynucleotide sequence encoding an Itch mutant for use as a medicament for treatment of various clinical stages of AD as described herein.

### BRIEF DESCRIPTION OF THE DRAWINGS

FIG. 1 shows the plasmids and the co-transfection of pAAV, pAAV2/9n and pAdDeltaF6 plasmids in HEK293 packaging cells results in formation of encapsulated recombinant AAVs.

FIG. 2A shows the fluorescence microscope images of HEK293T cells transduced with the purified AAVs.

FIG. 2B shows immunoblot of the lysates obtained from primary rat cortical neurons transduced with the purified AAVs as indicated and treated with AB42. After 48 h the lysates were prepared and subjected to immunoblotting with the indicated antibodies and β-actin was used as loading control.

FIG. 3 shows a schematic representation of example of AAV stereotaxic injections to 6-month-old to female WT and TgAD mice injected stereotaxically in the cortex with AAV-Ctrl (AAV-tdTomato control virus expressing modified RFP only), AAV-Itch, AAV-TS/A Itch (phosphodeficient mutant T222 and S232 mutated to alanine), AAV-K/R Itch (autoubiquitination mutant K393 mutated to arginine).

FIG. 4A shows the results of the Morris water maze test of WT and TgAD mice treated with AAV stereotaxic injections for four days.

FIG. 4B shows the results of the Morris water maze test in WT and TgAD mice treated AAV stereotaxic injections on final day. (Data represents as mean±SEM. * p<0.05, ns-not significant by one-way ANOVA followed by Tukey's post hoc test).

FIG. 4C shows representative track plots of WT and TgAD mice treated with different AAVs from the Morris water maze test.

FIG. 4D shows the results of the Morris water maze test in WT and TgAD mice in target crossing times in the area of hidden platform in the probe test (without platform) (Data presented as mean±SEM. * p<0.05, ns-not significant by one-way ANOVA test).

FIG. 5A shows the results of memory test in WT and TgAD mice from the Y-maze test.

FIG. 5B is the graphical representation of the total number of entries of each group in all arms of Y-maze. (Data represented as mean±SEM, not significant by one-way ANOVA).

FIG. 6A shows the Western blots obtained from the cortex tissue lysates of the brain WT and TgAD mice after behavior test.

FIG. 6B shows the quantification of the levels of TAp73 from the Western blot of FIG. 6A.

FIG. 7A shows fluorescence microscope images of PCNA (S phase)/NeuN assay along with Tunnel assay on cortex of WT and TgAD mouse.

FIG. 7B shows the quantification of the percent cell number for the TUNEL+PCNA+NeuN+ assay in FIG. 7A (Data presented as mean±SEM of N=3 mice per group, n=3 sections).

### BRIEF DESCRIPTION OF THE SEQUENCE LISTING

SEQ ID NO. 1 is the sequence of an AAV vector comprising the mouse nucleic acid sequence of wild type Itch.

SEQ ID NO. 2 is the sequence of an AAV vector comprising the mouse nucleic acid sequence encoding the T222/S232A Itch Phospho-deficient mutant.

SEQ ID NO. 3 is the sequence of an AAV vector comprising the mouse nucleic acid sequence encoding the K393R Itch Ubiquitination site mutant.

SEQ ID NO. 4 is the nucleic acid sequence of an AAV packaging plasmid expressing Rep/Cap genes (pAAV2/9n).

SEQ ID NO. 5 is the nucleic acid sequence of an AAV helper plasmid (pAdDeltaF6).

SEQ ID NO. 6 is the mouse amino acid sequence of wild type Itch.

SEQ ID NO. 7 is the mouse amino acid sequence of the T222/S232A Itch Phospho-deficient mutant.

SEQ ID NO. 8 is the mouse amino acid sequence of the K393R Itch Ubiquitination site mutant.

SEQ ID NO. 9 is the human amino acid sequence of wild-type Itch.

SEQ ID NO. 10 is the human nucleic acid sequence of wild-type Itch.

SEQ ID NO. 11 is the human amino acid sequence of the T222/S232A Itch Phospho-deficient mutant.

SEQ ID NO. 12 is the human nucleic acid sequence encoding the T222/S232A Itch Phospho-deficient mutant.

SEQ ID NO. 13 is the human amino acid sequence of the K391R Itch Ubiquitination site mutant.

SEQ ID NO. 14 is the human nucleic acid sequence encoding the K391R Itch Ubiquitination site mutant.

SEQ ID NO. 15 is the forward primer sequence employed to introduce the T222A mutation in the mouse Itch sequence.

SEQ ID NO. 16 is the reverse primer sequence employed to introduce the T222A mutation in the mouse Itch sequence.

SEQ ID NO. 17 is the forward primer sequence employed to introduce the S232A mutation in the mouse Itch sequence.

SEQ ID NO. 18 is the reverse primer sequence employed to introduce the S232A mutation in the mouse Itch sequence.

SEQ ID NO. 19 is the forward primer sequence employed to introduce the K393R mutation in the mouse Itch sequence.

SEQ ID NO. 20 is the reverse primer sequence employed to introduce the K393R mutation in the mouse Itch sequence.

### DETAILED DESCRIPTION OF THE DISCLOSURE

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having”, or “including but not limited to” wherever used, will be understood to imply the inclusion of a stated element, integer or step, or group of elements, integers or steps, but not the exclusion of any other element, integer or step, or group of elements, integers or steps.

Reference throughout this specification to “one embodiment”, “an embodiment”, or “some embodiments” means that a particular feature, structure or characteristic described in connection with the embodiment may be included in at least one embodiment of the present disclosure. Thus, the appearances of the phrases “in one embodiment”, “in an embodiment”, or “in some embodiments” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The term “subject” or “patient” as used herein refers to any mammal including, without limitation, humans and other primates (e.g., chimpanzees and other apes and monkey species), farm animals (e.g., cattle, sheep, pigs, goats and horses), domestic mammals (e.g., dogs and cats), and laboratory animals (e.g., rodents such as mice, rats, and guinea pigs). In some embodiments, the patient is a mammal. In some embodiments, the patient is a human.

Cell cycle suppression is an essential aspect in during differentiation of neurons. This post mitotic state of differentiated neurons can be altered by growth factor withdrawal or toxic insults by accumulation of misfolded proteins (Park, Morris et al. 1998). This alteration results in reactivation of the cell cycle leading to death of neurons. During cell cycle re-entry in AD, aberrant increase in cyclin D1 expression due to hyperactivation of MEK-ERK pathway results in S-phase progression (Modi, Komaravelli et al. 2012). In differentiated neurons the microRNA-34a (miR-34a) regulates cyclin D1 and TAp73 is a transcriptional activator of miR-34a. In AD mouse model neurons, the Aβ induced neurotoxicity causes TAp73 degradation resulting in disruption of miR-34a-cyclin D1 regulation and CRNA (Modi, Jaiswal et al. 2016). The present inventors found that Itch can be used as a therapeutic target to prevent aberrant activation of the cell cycle by degradation of TAp73 which induces the process of Cell Cycle Related Neuronal Apoptosis (CRNA). The inventors found that Itch mutants can restore the levels of TAp73 and reduce CRNA thereby inhibiting or reducing neurodegeneration observed in neural diseases. Further, the inventors observed that the administration of a viral vector expressing T222A and S232A mutant of Itch or K393R Itch mutant is able to rescue short- and long-term memory defects and improves memory and cognitive function in a mouse model of AD.

The present disclosure provides a method for treating a cognitive impairment due to Itch dysregulation in a patient, comprising administering to the patient a viral vector comprising a polynucleotide sequence encoding an Itch mutant.

In some embodiments, the method of treatment comprises administering a viral vector comprising a polynucleotide sequence encoding a mutation of Itch selected from the group consisting of: T222A, S232A, K393R and a combination thereof. In some embodiments, the method of treatment comprises administering a viral vector comprising a polynucleotide sequence encoding a mutation of Itch selected from the group consisting of: T222A, S232A, K391R and a combination thereof.

In some embodiments, the Itch mutant comprises T222A and S232A mutations. This mutant form of Itch is referred to herein as a phospho-deficient mutant as the T222 and S232 residues of Itch cannot be phosphorylated in this mutant. The position of phosphorylation residues T222 and S232 is the same in the mouse and human Itch proteins.

In some embodiments, the Itch mutant comprises K393R or K391R mutation. This mutant form of Itch is referred to herein as a ubiquitination site mutant as the K393 residue of Itch in mouse or K391 residue of Itch in human cannot be ubiquitinylated in this mutant.

In some embodiments, the polynucleotide sequences encoding Itch mutants are selected from Table A below:

TABLE A

| SEQ ID NO. | Nucleotide Sequence |
|---|---|
| SEQ ID NO: 2 | cttccgcttcctcgctcactgactcgctgcgctcggtcgttcggctgcggcgagcggtatcagctcactcaa |
| (Mouse) | aggcggtaatacggttatccacagaatcaggggataacgcaggaaagaacatgtgagcaaaaggccag |
| Nucleic acid | caaaaggccaggaaccgtaaaaaggccgcgttgctggcgtttttccataggctccgcccccctgacgagc |
| sequence | atcacaaaaatcgacgctcaagtcagaggtggcgaaacccgacaggactataaagataccaggcgtttcc |
| encoding T222A | ccctggaagctccctcgtgcgctctcctgttccgaccctgccgcttaccggatacctgtccgcctttctccctt |
| and S232A | cgggaagcgtggcgctttctcatagctcacgctgtaggtatctcagttcggtgtaggtcgttcgctccaagct |
| mutant | gggctgtgtgcacgaaccccccgttcagcccgaccgctgcgccttatccggtaactatcgtcttgagtcca |
| | acccggtaagacacgacttatcgccactggcagcagccactggtaacaggattagcagagcgaggtatgt |
| | aggcggtgctacagagttcttgaagtggtggcctaactacggctacactagaagaacagtatttggtatctg |
| | cgctctgctgaagccagttaccttcggaaaaagagttggtagctcttgatccggcaaacaaaccaccgctg |
| | gtagcggtggtttttttgtttgcaagcagcagattacgcgcagaaaaaaaggatctcaagaagatcctttgat |
| | cttttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaacttggt |
| | ctgacagttaccaatgcttaatcagtgaggcacctatctcagcgatctgtctatttcgttcatccatagttgcct |
| | gactccccgtcgtgtagataactacgatacgggagggcttaccatctggccccagtgctgcaatgataccg |
| | cgagacccacgctcaccggctccagatttatcagcaataaaccagccagccggaagggccgagcgcag |
| | aagtggtcctgcaactttatccgcctccatccagtctattaattgttgccgggaagctagagtaagtagttcgc |
| | cagttaatagtttgcgcaacgttgttgccattgctacaggcatcgtggtgtcacgctcgtcgtttggtatggctt |
| | cattcagctccggttcccaacgatcaaggcgagttacatgatcccccatgttgtgcaaaaaagcggttagct |
| | ccttcggtcctccgatcgttgtcagaagtaagttggccgcagtgttatcactcatggttatggcagcactgca |
| | taattctcttactgtcatgccatccgtaagatgcttttctgtgactggtgagtactcaaccaagtcattctgagaa |
| | tagtgtatgcggcgaccgagttgctcttgcccggcgtcaatacgggataataccgcgccacatagcagaa |
| | ctttaaaagtgctcatcattggaaaacgttcttcggggcgaaaactctcaaggatcttaccgctgttgagatc |
| | cagttcgatgtaacccactcgtgcacccaactgatcttcagcatcttttactttcaccagcgtttctgggtgag |
| | caaaaacaggaaggcaaaatgccgcaaaaaagggaataagggcgacacggaaatgttgaatactcata |
| | ctcttcctttttcaatattattgaagcatttatcagggttattgtctcatgagcggatacatatttgaatgtattta |
| | gaaaaataaacaaataggggttccgcgcacatttccccgaaaagtgccacctaaattgtaagcgttaatattttg |
| | ttaaaattcgcgttaaatttttgttaaatcagctcattttttaaccaataggccgaaatcggcaaaatcccttataa |
| | atcaaaagaatagaccgagatagggttgagtgttgttccagtttggaacaagagtccactattaaagaacgt |
| | ggactccaacgtcaaagggcgaaaaaccgtctatcagggcgatggcccactacgtgaaccatcacccta |
| | atcaagttttttggggtcgaggtgccgtaaagcactaaatcggaaccctaaagggagcccccgatttagag |
| | cttgacggggaaagccggcgaacgtggcgagaaaggaagggaagaaagcgaaaggagcgggcgct |
| | agggcgctggcaagtgtagcggtcacgctgcgcgtaaccaccacacccgccgcgcttaatgcgccgcta |
| | cagggcgcgtcccattcgccattcaggctgcgcaactgttgggaagggcgatcggtgcgggcctcttcgc |
| | tattacgccagctgcgcgctcgctcgctcactgaggccgcccgggcaaagcccgggcgtcgggcgacc |
| | tttggtcgcccggcctcagtgagcgagcgagcgcgcagagagggagtggccaactccatcactagggg |
| | ttccttgtagttaatgattaacccgccatgctacttatctacgtagccatgctctaggaagatcgtaccattgac |
| | gtcaataatgacgtatgttcccatagtaacgccaatagggactttccattgacgtcaatgggtggagtatttac |
| | ggtaaactgcccacttggcagtacatcaagtgtatcatatgccaagtacgccccctattgacgtcaatgacg |
| | gtaaatggcccgcctggcattatgcccagtacatgaccttatgggactttcctacttggcagtacatctacgt |
| | attagtcatcgctattaccatggtcgaggtgagccccacgttctgcttcactctccccatctcccccccctccc |
| | cacccccaattttgtatttatttattttttaattattttgtgcagcgatgggggcggggggggggggggggcg |
| | cgcgccaggcggggcggggcggggcgaggggcggggcggggcgaggcggagaggtgcggcggc |
| | agccaatcagagcggcgcgctccgaaagtttccttttatggcgaggcggcggcggcggcggccctataa |
| | aaagcgaagcgcgcggggggggagtcgctgcgacgctgccttcgccccgtgccccgctccgccgc |
| | cgcctcgcgccgcccgccccggctctgactgaccgcgttactcccacaggtgagcggggggacggcc |
| | cttctcctccgggctgtaattagcgcttggtttaatgacggcttgtttcttttctgtggctgcgtgaaagccttga |
| | ggggctccgggagggccctttgtgcggggggagcggctcggggctgtccgcggggggacggctgcct |
| | tcgggggggacggggcagggcggggttcggcttctggcgtgtgaccggcggctctagagcctctgcta |
| | accatgttcatgccttcttctttttcctacagctcctgggcaacgtgctggttattgtgctgtctcatcattttggc |
| | aaagaattggatccgccaccatggtgATGGAGCAGAAGCTGATTTCCGAGGA |
| | GGACCTGAACGGTGGTAGTCTGACCATGAAATCTCAACTTCAG |
| | ATCACTGTCATCTCAGCCAAACTTAAAGAAAATAAAAAGAATT |
| | GGTTTGGACCAAGTCCTTATGTAGAAGTCACAGTAGATGGACAG |
| | TCAAAGAAGACAGAAAAATGCAATAATACAAACAGTCCCAAGT |
| | GGAAGCAGCCCCTCACAGTTATTGTTACCCCTACGAGTAAATTA |
| | TGTTTTCGTGTGTGGAGTCACCAGACCCTGAAGTCTGATGTTTTA |
| | TTGGGAACTGCTGGATTAGATATTTATGAAACATTAAAGTCAAA |
| | CAATATGAAACTTGAAGAAGTAGTTATGACTTTGCAGCTTGTAG |
| | GTGACAAAGAGCCAACAGAGACGATGGGAGATTTGTCAGTTTG |
| | TCTTGATGGGCTGCAAGTAGAAGCTGAGGTTGTTACTAACGGTG |
| | AAACGTCATGCTCCGAGAGTACTACACAGAATGATGATGGCTG |
| | CAGAACCAGAGATGATACAAGAGTGAGCACAAATGGATCAGAG |
| | GACCCTGAGGTTGCAGCGTCAGGGGAAAACAAGAGGGCCAATG |
| | GGAACAATTCTCCGTCCCTTTCAAATGGTGGTTTTAAGCCTTCTA |
| | GACCTCCTAGACCTTCGAGACCACCTCCACCCGCTCCACGAAGA |
| | CCAGCTTCTGTCAATGGCGCACCATCCACGAATTCTGACAGTGA |
| | TGGATCTAGTACAGGCTCCTTGCCACCAACAAATACAAATGTAA |
| | ATACAAGTACATCTGAAGGAGCAACATCTGGATTAATAATTCCT |
| | CTTACTATATCTGGAGGCTCGGGCCCTAGGCCTCTGAATACTGT |
| | AAGCCAAGCTCCCCTACCACCTGGGTGGGAGCAGAGAGTAGAC |
| | CAGCATGGGCGTGTTTACTATGTAGACCATGTTGAAAAGCGAAC |
| | AACATGGGATAGACCAGAACCTCTACCTCCTGGCTGGGAACGG |
| | CGTGTGGACAATATGGGACGTATTTATTATGTTGATCATTTCAC |
| | AAGAACCACAACATGGCAGAGGCCAACCTTGGAATCTGTCCGG |
| | AACTATGAACAGTGGCAGCTACAGCGTAGTCAGCTTCAGGGAG |
| | CAATGCAGCAGTTTAACCAGAGATTCATCTATGGGAATCAAGAT |
| | TTGTTTGCTACATCACAAAACAAAGAATTTGATCCGCTTGGCCC |
| | GTTGCCCCCTGGATGGGAGAAGAGAACTGATAGCAATGGCAGA |
| | GTGTATTTTGTCAACCACAACACTCGGATTACTCAGTGGGAAGA |
| | CCCCAGAAGCCAAGGTCAGTTAAATGAAAAGCCCTTACCAGAA |
| | GGCTGGGAAATGAGATTCACAGTGGATGGAATTCCATATTTTGT |
| | GGACCACAATAGAAGAGCAACTACTTATATAGATCCACGAACG |
| | GGAAAATCAGCCTTAGACAATGGGCCCCAGATAGCCTATGTGC |
| | GGGACTTCAAGGCAAAAGTTCAGTATTTCCGGTTCTGGTGCCAG |
| | CAACTGGCCATGCCACAGCATATAAAGATCACAGTGACAAGAA |
| | AAACATTGTTTGAGGATTCCTTTCAGCAGATCATGAGCTTCAGC |
| | CCGCAAGACCTGAGAAGACGTTTGTGGGTGATTTTCCCAGGAGA |
| | AGAAGGTTTAGATTATGGAGGTGTAGCAAGAGAATGGTTCTTTC |
| | TTTTGTCACATGAAGTGTTGAACCCAATGTATTGCCTGTTTGAAT |
| | ATGCAGGGAAGGATAACTACTGCCTGCAGATAAACCCCGCTTCT |
| | TACATCAATCCAGACCACCTGAAATACTTTCGTTTTATTGGCAG |
| | ATTTATTGCCATGGCTCTGTTTCATGGGAAATTCATAGATACCG |
| | GATTTTCTTTACCATTCTATAAACGTATCCTGAATAAACCAGTTG |
| | GACTTAAGGATTTAGAATCTATTGATCCAGAATTTTATAATTCG |
| | CTCATCTGGGTTAAAGAAAACAACATCGAAGAATGTGGTTTGG |
| | AAATGTACTTCTCAGTTGATAAAGAAATTCTAGGTGAAATTAAG |
| | AGTCATGATTTGAAACCGAATGGTGGCAATATTCTTGTGACAGA |
| | AGAAAACAAGGAGGAATACATCAGGATGGTAGCTGAGTGGAGG |
| | TTGTCTCGAGGTGTTGAAGAACAGACACAAGCTTTCTTTGAGGG |
| | CTTTAATGAAATTCTTCCTCAGCAATATTTGCAATATTTTGATGC |
| | GAAGGAATTAGAGGTTCTTCTGTGTGGGATGCAGGAGATTGATT |
| | TGAATGACTGGCAGAGACATGCCATCTACCGCCACTACACCAG |
| | AACAAGCAAGCAGATCATGTGGTTTTGGCAGTTTGTTAAAGAAA |
| | TTGATAATGAGAAGAGGATGAGACTTCTGCAGTTTGTTACTGGA |
| | ACCTGCCGATTGCCAGTGGGAGGATTTGCTGACCTTATGGGGAG |
| | TAATGGACCACAGAAGTTCTGCATCGAAAAAGTTGGCAAAGAA |
| | AATTGGTTACCCAGAAGCCATACTTGTTTTAACCGCCTGGACCT |
| | CCCACCTTACAAGAGCTATGAGCAACTGAAGGAAAAGCTGTTA |
| | TTTGCCATTGAAGAAACTGAAGGATTTGGACAAGAGggatccggtacc |
| | gccaccatggtgagtaagggcgaggaagtgatcaaagagttcatgcggtttaaggtgagaatggaagga |
| | agcatgaacggccacgagttcgaaattgagggagaaggagagggacggccctacgagggcacccaga |
| | cagccaagctgaaagtgacaaaggggggcctctgccattcgcttgggacatcctgagcccacagtttat |
| | gtacggctccaaggcctatgtgaaacatccagctgacattcccgattataagaaactgagcttccccgagg |
| | ggtttaagtgggaaagagtgatgaacttcgaggacggaggcctggtgactgtgacccaggacagctccct |
| | gcaggatgggaccctgatctacaaggtgaaaatgagagggacaaattttccccctgatggacctgtgatgc |
| | agaagaaaactatgggatgggaggcctccaccgaaaggctgtatccacgcgacggggtgctgaaagga |
| | gaaatccaccaggctctgaagctgaaagatgggggacattacctggtggagttcaagacaatctacatgg |
| | ccaagaaacctgtgcagctgccaggctactattacgtggacacaaaactggatatcacttcacacaacgag |
| | gactacactattgtggagcagtatgaacggagcgaggggagacaccatctgttcctgggccatgggactg |
| | gaagtaccggctcagggtctagtggaaccgcctcaagcgaggataacaatatggctgtgatcaaagagtt |
| | catgaggtttaaggtgcgcatggagggcagcatgaatgggcacgaatttgagattgaaggagagggcga |
| | agggaggccttacgagggcacacagactgccaagctgaaagtgaccaagggaggaccactgcctttcg |
| | cttgggatatcctgtctcctcagtttatgtacggaagtaaggcctatgtcaagcatcccgctgacattcctgatt |
| | acaagaaactgtctttcccagagggctttaagtgggagagagtgatgaattttgaagatggaggcctggtg |
| | accgtgacacaggactcctctctgcaggatggcactctgatctacaaagtcaaaatgcgcggcaccaatttt |
| | ccacccgatgggcccgtgatgcagaagaaaacaatggggtgggaggccagcactgaacggctgtatcc |
| | tagagacggagtgctgaagggcgaaatccaccaggccctgaagctgaaagacggcggccactacctgg |
| | tggagttcaaaaccatctacatggccaagaaaccagtgcagctgcccggctattactatgtggacaccaag |
| | ctggatatcacatcccacaatgaagactacaccattgtggaacagtatgagaggtctgaaggacgccacc |
| | atctgtttctgtacggcatggatgagctgtataagtaagaattcgatatcaagcttatcgataatcaacctctgg |
| | attacaaaatttgtgaaagattgactggtattcttaactatgttgctccttttacgctatgtggatacgctgcttta |
| | atgcctttgtatcatgctattgcttcccgtatggctttcattttctcctccttgtataaatcctggttgctgtctct |
| | tgaggagttgtggcccgttgtcaggcaacgtggcgtggtgtgcactgtgtttgctgacgcaacccccactg |
| | ttagttggggcattgccaccacctgtcagctcctttccgggactttcgctttccccctccctattgccacggcgga |
| | actcatcgccgcctgccttgcccgctgctggacaggggctcggctgttgggcactgacaattccgtggtgt |
| | tgtcggggaaatcatcgtcctttccttggctgctcgcctgtgttgccacctggattctgcgcgggacgtcctt |
| | ctgctacgtcccttcggccctcaatccagcggaccttccttcccgcggcctgctgccggctctgcggcctct |
| | tccgcgtcttcgccttcgccctcagacgagtcggatctccctttgggccgcctccccgcatcgataccgtcg |
| | acccgggcggccgcttcgagcagacatgataagatacattgatgagtttggacaaaccacaactagaatg |
| | cagtgaaaaaaatgctttatttgtgaaatttgtgatgctattgctttatttgtaaccattataagctgcaataaaca |
| | agttaacaacaacaattgcattcattttatgtttcaggttcagggggagatgtgggaggttttttaaagcaagt |
| | aaaacctctacaaatgtggtaaaatcgataaggatcttcctagagcatggctacgtagataagtagcatggc |
| | gggttaatcattaactacaaggaacccctagtgatggagttggccactccctctctgcgcgctcgctcgctc |
| | actgaggccgggcgaccaaaggtcgcccgacgcccgggctttgcccgggcggcctcagtgagcgagc |
| | gagcgcgcagctgcattaatgaatcggccaacgcgcggggagaggcggtttgcgtattgggcgct |
| | Bold and underlined-myc-tag |
| | Bold-T222A and S232A mutation nucleotides |
| SEQ ID NO: 3 | cttccgcttcctcgctcactgactcgctgcgctcggtcgttcggctgcggcgagcggtatcagctcactcaa |
| (Mouse) Nucleic | aggcggtaatacggttatccacagaatcaggggataacgcaggaaagaacatgtgagcaaaaggccag |
| acid sequence | caaaaggccaggaaccgtaaaaaggccgcgttgctggcgtttttccataggctccgcccccctgacgagc |
| encoding K393R | atcacaaaaatcgacgctcaagtcagaggtggcgaaacccgacaggactataaagataccaggcgtttcc |
| Itch mutant | ccctggaagctccctcgtgcgctctcctgttccgaccctgccgcttaccggatacctgtccgcctttctccctt |
| | cgggaagcgtggcgctttctcatagctcacgctgtaggtatctcagttcggtgtaggtcgttcgctccaagct |
| | gggctgtgtgcacgaaccccccgttcagcccgaccgctgcgccttatccggtaactatcgtcttgagtcca |
| | acccggtaagacacgacttatcgccactggcagcagccactggtaacaggattagcagagcgaggtatgt |
| | aggcggtgctacagagttcttgaagtggtggcctaactacggctacactagaagaacagtatttggtatctg |
| | cgctctgctgaagccagttaccttcggaaaaagagttggtagctcttgatccggcaaacaaaccaccgctg |
| | gtagcggtggtttttttgtttgcaagcagcagattacgcgcagaaaaaaaggatctcaagaagatcctttgat |
| | cttttctacggggtctgacgctcagtggaacgaaaactcacgttaagggattttggtcatgagattatcaaaa |
| | aggatcttcacctagatccttttaaattaaaaatgaagttttaaatcaatctaaagtatatatgagtaaacttggt |
| | ctgacagttaccaatgcttaatcagtgaggcacctatctcagcgatctgtctatttcgttcatccatagttgcct |
| | gactccccgtcgtgtagataactacgatacgggagggcttaccatctggccccagtgctgcaatgataccg |
| | cgagacccacgctcaccggctccagatttatcagcaataaaccagccagccggaagggccgagcgcag |
| | aagtggtcctgcaactttatccgcctccatccagtctattaattgttgccgggaagctagagtaagtagttcgc |
| | cagttaatagtttgcgcaacgttgttgccattgctacaggcatcgtggtgtcacgctcgtcgtttggtatggctt |
| | cattcagctccggttcccaacgatcaaggcgagttacatgatcccccatgttgtgcaaaaaagcggttagct |
| | ccttcggtcctccgatcgttgtcagaagtaagttggccgcagtgttatcactcatggttatggcagcactgca |
| | taattctcttactgtcatgccatccgtaagatgcttttctgtgactggtgagtactcaaccaagtcattctgagaa |
| | tagtgtatgcggcgaccgagttgctcttgcccggcgtcaatacgggataataccgcgccacatagcagaa |
| | ctttaaaagtgctcatcattggaaaacgttcttcggggcgaaaactctcaaggatcttaccgctgttgagato |
| | cagttcgatgtaacccactcgtgcacccaactgatcttcagcatcttttactttcaccagcgtttctgggtgag |
| | caaaaacaggaaggcaaaatgccgcaaaaaagggaataagggcgacacggaaatgttgaatactcata |
| | ctcttcctttttcaatattattgaagcatttatcagggttattgtctcatgagcggatacatatttgaatgtattta |
| | gaaaaataaacaaataggggttccgcgcacatttccccgaaaagtgccacctaaattgtaagcgttaatattttg |
| | ttaaaattcgcgttaaatttttgttaaatcagctcattttttaaccaataggccgaaatcggcaaaatcccttataa |
| | atcaaaagaatagaccgagatagggttgagtgttgttccagtttggaacaagagtccactattaaagaacgt |
| | ggactccaacgtcaaagggcgaaaaaccgtctatcagggcgatggcccactacgtgaaccatcacccta |
| | atcaagttttttggggtcgaggtgccgtaaagcactaaatcggaaccctaaagggagcccccgatttagag |
| | cttgacggggaaagccggcgaacgtggcgagaaaggaagggaagaaagcgaaaggagcgggcgct |
| | agggcgctggcaagtgtagcggtcacgctgcgcgtaaccaccacacccgccgcgcttaatgcgccgcta |
| | cagggcgcgtcccattcgccattcaggctgcgcaactgttgggaagggcgatcggtgcgggcctcttcgc |
| | tattacgccagctgcgcgctcgctcgctcactgaggccgcccgggcaaagcccgggcgtcgggcgacc |
| | tttggtcgcccggcctcagtgagcgagcgagcgcgcagagagggagtggccaactccatcactagggg |
| | ttccttgtagttaatgattaacccgccatgctacttatctacgtagccatgctctaggaagatcgtaccattgac |
| | gtcaataatgacgtatgttcccatagtaacgccaatagggactttccattgacgtcaatgggtggagtatttac |
| | ggtaaactgcccacttggcagtacatcaagtgtatcatatgccaagtacgccccctattgacgtcaatgacg |
| | gtaaatggcccgcctggcattatgcccagtacatgaccttatgggactttcctacttggcagtacatctacgt |
| | attagtcatcgctattaccatggtcgaggtgagccccacgttctgcttcactctccccatctcccccccctccc |
| | cacccccaattttgtatttatttattttttaattattttgtgcagcgatgggggcggggggggggggggggcg |
| | cgcgccaggcggggcggggcggggcgaggggcggggcggggcgaggcggagaggtgcggcggc |
| | agccaatcagagcggcgcgctccgaaagtttccttttatggcgaggcggcggcggcggcggccctataa |
| | aaagcgaagcgcgcggggggggagtcgctgcgacgctgccttcgccccgtgccccgctccgccgc |
| | cgcctcgcgccgcccgccccggctctgactgaccgcgttactcccacaggtgagcgggcgggacggcc |
| | cttctcctccgggctgtaattagcgcttggtttaatgacggcttgtttcttttctgtggctgcgtgaaagccttga |
| | ggggctccgggagggccctttgtgcggggggagcggctcggggctgtccgcggggggacggctgcct |
| | tcgggggggacggggcagggcggggttcggcttctggcgtgtgaccggcggctctagagcctctgcta |
| | accatgttcatgccttcttctttttcctacagctcctgggcaacgtgctggttattgtgctgtctcatcattttggc |
| | aaagaattggatccgccaccatggtgATGGAGCAGAAGCTGATTTCCGAGGA |
| | GGACCTGAACGGTGGTAGTCTGACCATGAAATCTCAACTTCAG |
| | ATCACTGTCATCTCAGCCAAACTTAAAGAAAATAAAAAGAATT |
| | GGTTTGGACCAAGTCCTTATGTAGAAGTCACAGTAGATGGACAG |
| | TCAAAGAAGACAGAAAAATGCAATAATACAAACAGTCCCAAGT |
| | GGAAGCAGCCCCTCACAGTTATTGTTACCCCTACGAGTAAATTA |
| | TGTTTTCGTGTGTGGAGTCACCAGACCCTGAAGTCTGATGTTTTA |
| | TTGGGAACTGCTGGATTAGATATTTATGAAACATTAAAGTCAAA |
| | CAATATGAAACTTGAAGAAGTAGTTATGACTTTGCAGCTTGTAG |
| | GTGACAAAGAGCCAACAGAGACGATGGGAGATTTGTCAGTTTG |
| | TCTTGATGGGCTGCAAGTAGAAGCTGAGGTTGTTACTAACGGTG |
| | AAACGTCATGCTCCGAGAGTACTACACAGAATGATGATGGCTG |
| | CAGAACCAGAGATGATACAAGAGTGAGCACAAATGGATCAGAG |
| | GACCCTGAGGTTGCAGCGTCAGGGGAAAACAAGAGGGCCAATG |
| | GGAACAATTCTCCGTCCCTTTCAAATGGTGGTTTTAAGCCTTCTA |
| | GACCTCCTAGACCTTCGAGACCACCTCCACCCACCCCACGAAGA |
| | CCAGCTTCTGTCAATGGCTCACCATCCACGAATTCTGACAGTGA |
| | TGGATCTAGTACAGGCTCCTTGCCACCAACAAATACAAATGTAA |
| | ATACAAGTACATCTGAAGGAGCAACATCTGGATTAATAATTCCT |
| | CTTACTATATCTGGAGGCTCGGGCCCTAGGCCTCTGAATACTGT |
| | AAGCCAAGCTCCCCTACCACCTGGGTGGGAGCAGAGAGTAGAC |
| | CAGCATGGGCGTGTTTACTATGTAGACCATGTTGAAAAGCGAAC |
| | AACATGGGATAGACCAGAACCTCTACCTCCTGGCTGGGAACGG |
| | CGTGTGGACAATATGGGACGTATTTATTATGTTGATCATTTCAC |
| | AAGAACCACAACATGGCAGAGGCCAACCTTGGAATCTGTCCGG |
| | AACTATGAACAGTGGCAGCTACAGCGTAGTCAGCTTCAGGGAG |
| | CAATGCAGCAGTTTAACCAGAGATTCATCTATGGGAATCAAGAT |
| | TTGTTTGCTACATCACAAAACAGAGAATTTGATCCGCTTGGCCC |
| | GTTGCCCCCTGGATGGGAGAAGAGAACTGATAGCAATGGCAGA |
| | GTGTATTTTGTCAACCACAACACTCGGATTACTCAGTGGGAAGA |
| | CCCCAGAAGCCAAGGTCAGTTAAATGAAAAGCCCTTACCAGAA |
| | GGCTGGGAAATGAGATTCACAGTGGATGGAATTCCATATTTTGT |
| | GGACCACAATAGAAGAGCAACTACTTATATAGATCCACGAACG |
| | GGAAAATCAGCCTTAGACAATGGGCCCCAGATAGCCTATGTGC |
| | GGGACTTCAAGGCAAAAGTTCAGTATTTCCGGTTCTGGTGCCAG |
| | CAACTGGCCATGCCACAGCATATAAAGATCACAGTGACAAGAA |
| | AAACATTGTTTGAGGATTCCTTTCAGCAGATCATGAGCTTCAGC |
| | CCGCAAGACCTGAGAAGACGTTTGTGGGTGATTTTCCCAGGAGA |
| | AGAAGGTTTAGATTATGGAGGTGTAGCAAGAGAATGGTTCTTTC |
| | TTTTGTCACATGAAGTGTTGAACCCAATGTATTGCCTGTTTGAAT |
| | ATGCAGGGAAGGATAACTACTGCCTGCAGATAAACCCCGCTTCT |
| | TACATCAATCCAGACCACCTGAAATACTTTCGTTTTATTGGCAG |
| | ATTTATTGCCATGGCTCTGTTTCATGGGAAATTCATAGATACCG |
| | GATTTTCTTTACCATTCTATAAACGTATCCTGAATAAACCAGTTG |
| | GACTTAAGGATTTAGAATCTATTGATCCAGAATTTTATAATTCG |
| | CTCATCTGGGTTAAAGAAAACAACATCGAAGAATGTGGTTTGG |
| | AAATGTACTTCTCAGTTGATAAAGAAATTCTAGGTGAAATTAAG |
| | AGTCATGATTTGAAACCGAATGGTGGCAATATTCTTGTGACAGA |
| | AGAAAACAAGGAGGAATACATCAGGATGGTAGCTGAGTGGAGG |
| | TTGTCTCGAGGTGTTGAAGAACAGACACAAGCTTTCTTTGAGGG |
| | CTTTAATGAAATTCTTCCTCAGCAATATTTGCAATATTTTGATGC |
| | GAAGGAATTAGAGGTTCTTCTGTGTGGGATGCAGGAGATTGATT |
| | TGAATGACTGGCAGAGACATGCCATCTACCGCCACTACACCAG |
| | AACAAGCAAGCAGATCATGTGGTTTTGGCAGTTTGTTAAAGAAA |
| | TTGATAATGAGAAGAGGATGAGACTTCTGCAGTTTGTTACTGGA |
| | ACCTGCCGATTGCCAGTGGGAGGATTTGCTGACCTTATGGGGAG |
| | TAATGGACCACAGAAGTTCTGCATCGAAAAAGTTGGCAAAGAA |
| | AATTGGTTACCCAGAAGCCATACTTGTTTTAACCGCCTGGACCT |
| | CCCACCTTACAAGAGCTATGAGCAACTGAAGGAAAAGCTGTTA |
| | TTTGCCATTGAAGAAACTGAAGGATTTGGACAAGAGggatccggtacc |
| | gccaccatggtgagtaagggcgaggaagtgatcaaagagttcatgcggtttaaggtgagaatggaagga |
| | agcatgaacggccacgagttcgaaattgagggagaaggagagggacggccctacgagggcacccaga |
| | cagccaagctgaaagtgacaaaggggggcctctgccattcgcttgggacatcctgagcccacagtttat |
| | gtacggctccaaggcctatgtgaaacatccagctgacattcccgattataagaaactgagcttccccgagg |
| | ggtttaagtgggaaagagtgatgaacttcgaggacggaggcctggtgactgtgacccaggacagctccct |
| | gcaggatgggaccctgatctacaaggtgaaaatgagagggacaaattttccccctgatggacctgtgatgc |
| | agaagaaaactatgggatgggaggcctccaccgaaaggctgtatccacgcgacggggtgctgaaagga |
| | gaaatccaccaggctctgaagctgaaagatgggggacattacctggtggagttcaagacaatctacatgg |
| | ccaagaaacctgtgcagctgccaggctactattacgtggacacaaaactggatatcacttcacacaacgag |
| | gactacactattgtggagcagtatgaacggagcgaggggagacaccatctgttcctgggccatgggactg |
| | gaagtaccggctcagggtctagtggaaccgcctcaagcgaggataacaatatggctgtgatcaaagagtt |
| | catgaggtttaaggtgcgcatggagggcagcatgaatgggcacgaatttgagattgaaggagagggcga |
| | agggaggccttacgagggcacacagactgccaagctgaaagtgaccaagggaggaccactgcctttcg |
| | cttgggatatcctgtctcctcagtttatgtacggaagtaaggcctatgtcaagcatcccgctgacattcctgatt |
| | acaagaaactgtctttcccagagggctttaagtgggagagagtgatgaattttgaagatggaggcctggtg |
| | accgtgacacaggactcctctctgcaggatggcactctgatctacaaagtcaaaatgcgcggcaccaatttt |
| | ccacccgatgggcccgtgatgcagaagaaaacaatggggtgggaggccagcactgaacggctgtatcc |
| | tagagacggagtgctgaagggcgaaatccaccaggccctgaagctgaaagacggcggccactacctgg |
| | tggagttcaaaaccatctacatggccaagaaaccagtgcagctgcccggctattactatgtggacaccaag |
| | ctggatatcacatcccacaatgaagactacaccattgtggaacagtatgagaggtctgaaggacgccacc |
| | atctgtttctgtacggcatggatgagctgtataagtaagaattcgatatcaagcttatcgataatcaacctctgg |
| | attacaaaatttgtgaaagattgactggtattcttaactatgttgctccttttacgctatgtggatacgctgcttta |
| | atgcctttgtatcatgctattgcttcccgtatggctttcattttctcctccttgtataaatcctggttgctgtctcttta |
| | tgaggagttgtggcccgttgtcaggcaacgtggcgtggtgtgcactgtgtttgctgacgcaacccccactg |
| | gttggggcattgccaccacctgtcagctcctttccgggactttcgctttccccctccctattgccacggcgga |
| | actcatcgccgcctgccttgcccgctgctggacaggggctcggctgttgggcactgacaattccgtggtgt |
| | tgtcggggaaatcatcgtcctttccttggctgctcgcctgtgttgccacctggattctgcgcgggacgtcctt |
| | ctgctacgtcccttcggccctcaatccagcggaccttccttcccgcggcctgctgccggctctgcggcctct |
| | tccgcgtcttcgccttcgccctcagacgagtcggatctccctttgggccgcctccccgcategataccgtcg |
| | acccgggcggccgcttcgagcagacatgataagatacattgatgagtttggacaaaccacaactagaatg |
| | cagtgaaaaaaatgctttatttgtgaaatttgtgatgctattgctttatttgtaaccattataagctgcaataaaca |
| | agttaacaacaacaattgcattcattttatgtttcaggttcagggggagatgtgggaggttttttaaagcaagt |
| | aaaacctctacaaatgtggtaaaatcgataaggatcttcctagagcatggctacgtagataagtagcatggc |
| | gggttaatcattaactacaaggaacccctagtgatggagttggccactccctctctgcgcgctcgctegctc |
| | actgaggccgggcgaccaaaggtcgcccgacgcccgggctttgcccgggcggcctcagtgagcgagc |
| | gagcgcgcagctgcattaatgaatcggccaacgcgcggggagaggcggtttgcgtattgggcgct |
| | Bold and underlined-myc-tag |
| | Bold-K393R mutation nucleotides |
| SEQ ID NO: 12 | ATGAGCGACAGCGGCAGCCAGCTGGGCAGCATGGGCAGCCTGACCATGAAGAGCCAGCTG |
| (human nucleic | CAGATCACCGTGATCAGCGCCAAGCTGAAGGAGAACAAGAAGAACTGGTTCGGCCCCAGC |
| acid sequence | CCCTACGTGGAGGTGACCGTGGACGGCCAGAGCAAGAAGACCGAGAAGTGCAACAACACC |
| encoding the | AACAGCCCCAAGTGGAAGCAGCCCCTGACCGTGATCGTGACCCCCGTGAGCAAGCTGCAC |
| T222/S232A Itch | TTCAGGGTGTGGAGCCACCAGACCCTGAAGAGCGACGTGCTGCTGGGCACCGCCGCCCTG |
| Phospho- | GACATCTACGAGACCCTGAAGAGCAACAACATGAAGCTGGAGGAGGTGGTGGTGACCCTG |
| deficient | CAGCTGGGGGGCGACAAGGAGCCCACCGAGACCATCGGCGACCTGAGCATCTGCCTGGAC |
| mutant) | GGCCTGCAGCTGGAGAGCGAGGTGGTGACCAACGGCGAGACCACCTGCAGCGAGAGCGCC |
| | AGCCAGAACGACGACGGCAGCAGGAGCAAGGACGAGACCAGGGTGAGCACCAACGGCAGC |
| | GACGACCCCGAGGACGCCGGCGCCGGCGAGAACAGGAGGGTGAGCGGCAACAACAGCCCC |
| | AGCCTGAGCAACGGCGGCTTCAAGCCCAGCAGGCCCCCCAGGCCCAGCAGGCCCCCCCCC |
| | CCCGCTCCCAGGAGGCCCGCCAGCGTGAACGGCGCACCCAGCGCCACCAGCGAGAGCGAC |
| | GGCAGCAGCACCGGCAGCCTGCCCCCCACCAACACCAACACCAACACCAGCGAGGGCGCC |
| | ACCAGCGGCCTGATCATCCCCCTGACCATCAGCGGCGGCAGCGGCCCCAGGCCCCTGAAC |
| | CCCGTGACCCAGGCCCCCCTGCCCCCCGGCTGGGAGCAGAGGGTGGACCAGCACGGCAGG |
| | GTGTACTACGTGGACCACGTGGAGAAGAGGACCACCTGGGACAGGCCCGAGCCCCTGCCC |
| | CCCGGCTGGGAGAGGAGGGTGGACAACATGGGCAGGATCTACTACGTGGACCACTTCACC |
| | AGGACCACCACCTGGCAGAGGCCCACCCTGGAGAGCGTGAGGAACTACGAGCAGTGGCAG |
| | CTGCAGAGGAGCCAGCTGCAGGGCGCCATGCAGCAGTTCAACCAGAGGTTCATCTACGGC |
| | AACCAGGACCTGTTCGCCACCAGCCAGAGCAAGGAGTTCGACCCCCTGGGCCCCCTGCCC |
| | CCCGGCTGGGAGAAGAGGACCGACAGCAACGGCAGGGTGTACTTCGTGAACCACAACACC |
| | AGGATCACCCAGTGGGAGGACCCCAGGAGCCAGGGCCAGCTGAACGAGAAGCCCCTGCCC |
| | GAGGGCTGGGAGATGAGGTTCACCGTGGACGGCATCCCCTACTTCGTGGACCACAACAGG |
| | AGGACCACCACCTACATCGACCCCAGGACCGGCAAGAGCGCCCTGGACAACGGCCCCCAG |
| | ATCGCCTACGTGAGGGACTTCAAGGCCAAGGTGCAGTACTTCAGGTTCTGGTGCCAGCAG |
| | CTGGCCATGCCCCAGCACATCAAGATCACCGTGACCAGGAAGACCCTGTTCGAGGACAGC |
| | TTCCAGCAGATCATGAGCTTCAGCCCCCAGGACCTGAGGAGGAGGCTGTGGGTGATCTTC |
| | CCCGGCGAGGAGGGCCTGGACTACGGCGGCGTGGCCAGGGAGTGGTTCTTCCTGCTGAGC |
| | CACGAGGTGCTGAACCCCATGTACTGCCTGTTCGAGTACGCCGGCAAGGACAACTACTGC |
| | CTGCAGATCAACCCCGCCAGCTACATCAACCCCGACCACCTGAAGTACTTCAGGTTCATC |
| | GGCAGGTTCATCGCCATGGCCCTGTTCCACGGCAAGTTCATCGACACCGGCTTCAGCCTG |
| | CCCTTCTACAAGAGGATCCTGAACAAGCCCGTGGGCCTGAAGGACCTGGAGAGCATCGAC |
| | CCCGAGTTCTACAACAGCCTGATCTGGGTGAAGGAGAACAACATCGAGGAGTGCGACCTG |
| | GAGATGTACTTCAGCGTGGACAAGGAGATCCTGGGCGAGATCAAGAGCCACGACCTGAAG |
| | CCCAACGGCGGCAACATCCTGGTGACCGAGGAGAACAAGGAGGAGTACATCAGGATGGTG |
| | GCCGAGTGGAGGCTGAGCAGGGGCGTGGAGGAGCAGACCCAGGCCTTCTTCGAGGGCTTC |
| | AACGAGATCCTGCCCCAGCAGTACCTGCAGTACTTCGACGCCAAGGAGCTGGAGGTGCTG |
| | CTGTGCGGCATGCAGGAGATCGACCTGAACGACTGGCAGAGGCACGCCATCTACAGGCAC |
| | TACGCCAGGACCAGCAAGCAGATCATGTGGTTCTGGCAGTTCGTGAAGGAGATCGACAAC |
| | GAGAAGAGGATGAGGCTGCTGCAGTTCGTGACCGGCACCTGCAGGCTGCCCGTGGGGGGC |
| | TTCGCCGACCTGATGGGCAGCAACGGCCCCCAGAAGTTCTGCATCGAGAAGGTGGGCAAG |
| | GAGAACTGGCTGCCCAGGAGCCACACCTGCTTCAACAGGCTGGACCTGCCCCCCTACAAG |
| | AGCTACGAGCAGCTGAAGGAGAAGCTGCTGTTCGCCATCGAGGAGACCGAGGGCTTCGGC |
| | CAGGAGTAA |
| | Bold-T222A and S232A mutation nucleotides |
| SEQ ID NO: 14 | ATGAGCGACAGCGGCAGCCAGCTGGGCAGCATGGGCAGCCTGACCATGAAGAGCCAGCTG |
| (human nucleic | CAGATCACCGTGATCAGCGCCAAGCTGAAGGAGAACAAGAAGAACTGGTTCGGCCCCAGC |
| acid sequence | CCCTACGTGGAGGTGACCGTGGACGGCCAGAGCAAGAAGACCGAGAAGTGCAACAACACC |
| encoding the | AACAGCCCCAAGTGGAAGCAGCCCCTGACCGTGATCGTGACCCCCGTGAGCAAGCTGCAC |
| K391R Itch | TTCAGGGTGTGGAGCCACCAGACCCTGAAGAGCGACGTGCTGCTGGGCACCGCCGCCCTG |
| Ubiquitination | GACATCTACGAGACCCTGAAGAGCAACAACATGAAGCTGGAGGAGGTGGTGGTGACCCTG |
| site mutant) | CAGCTGGGCGGCGACAAGGAGCCCACCGAGACCATCGGCGACCTGAGCATCTGCCTGGAC |
| | GGCCTGCAGCTGGAGAGCGAGGTGGTGACCAACGGCGAGACCACCTGCAGCGAGAGCGCC |
| | AGCCAGAACGACGACGGCAGCAGGAGCAAGGACGAGACCAGGGTGAGCACCAACGGCAGC |
| | GACGACCCCGAGGACGCCGGCGCCGGCGAGAACAGGAGGGTGAGCGGCAACAACAGCCCC |
| | AGCCTGAGCAACGGCGGCTTCAAGCCCAGCAGGCCCCCCAGGCCCAGCAGGCCCCCCCCC |
| | CCCACCCCCAGGAGGCCCGCCAGCGTGAACGGCAGCCCCAGCGCCACCAGCGAGAGCGAC |
| | GGCAGCAGCACCGGCAGCCTGCCCCCCACCAACACCAACACCAACACCAGCGAGGGCGCC |
| | ACCAGCGGCCTGATCATCCCCCTGACCATCAGCGGCGGCAGCGGCCCCAGGCCCCTGAAC |
| | CCCGTGACCCAGGCCCCCCTGCCCCCCGGCTGGGAGCAGAGGGTGGACCAGCACGGCAGG |
| | GTGTACTACGTGGACCACGTGGAGAAGAGGACCACCTGGGACAGGCCCGAGCCCCTGCCC |
| | CCCGGCTGGGAGAGGAGGGTGGACAACATGGGCAGGATCTACTACGTGGACCACTTCACC |
| | AGGACCACCACCTGGCAGAGGCCCACCCTGGAGAGCGTGAGGAACTACGAGCAGTGGCAG |
| | CTGCAGAGGAGCCAGCTGCAGGGCGCCATGCAGCAGTTCAACCAGAGGTTCATCTACGGC |
| | AACCAGGACCTGTTCGCCACCAGCCAGAGCAGAGAGTTCGACCCCCTGGGCCCCCTGCCC |
| | CCCGGCTGGGAGAAGAGGACCGACAGCAACGGCAGGGTGTACTTCGTGAACCACAACACC |
| | AGGATCACCCAGTGGGAGGACCCCAGGAGCCAGGGCCAGCTGAACGAGAAGCCCCTGCCC |
| | GAGGGCTGGGAGATGAGGTTCACCGTGGACGGCATCCCCTACTTCGTGGACCACAACAGG |
| | AGGACCACCACCTACATCGACCCCAGGACCGGCAAGAGCGCCCTGGACAACGGCCCCCAG |
| | ATCGCCTACGTGAGGGACTTCAAGGCCAAGGTGCAGTACTTCAGGTTCTGGTGCCAGCAG |
| | CTGGCCATGCCCCAGCACATCAAGATCACCGTGACCAGGAAGACCCTGTTCGAGGACAGC |
| | TTCCAGCAGATCATGAGCTTCAGCCCCCAGGACCTGAGGAGGAGGCTGTGGGTGATCTTC |
| | CCCGGCGAGGAGGGCCTGGACTACGGCGGCGTGGCCAGGGAGTGGTTCTTCCTGCTGAGC |
| | CACGAGGTGCTGAACCCCATGTACTGCCTGTTCGAGTACGCCGGCAAGGACAACTACTGC |
| | CTGCAGATCAACCCCGCCAGCTACATCAACCCCGACCACCTGAAGTACTTCAGGTTCATC |
| | GGCAGGTTCATCGCCATGGCCCTGTTCCACGGCAAGTTCATCGACACCGGCTTCAGCCTG |
| | CCCTTCTACAAGAGGATCCTGAACAAGCCCGTGGGCCTGAAGGACCTGGAGAGCATCGAC |
| | CCCGAGTTCTACAACAGCCTGATCTGGGTGAAGGAGAACAACATCGAGGAGTGCGACCTG |
| | GAGATGTACTTCAGCGTGGACAAGGAGATCCTGGGCGAGATCAAGAGCCACGACCTGAAG |
| | CCCAACGGCGGCAACATCCTGGTGACCGAGGAGAACAAGGAGGAGTACATCAGGATGGTG |
| | GCCGAGTGGAGGCTGAGCAGGGGCGTGGAGGAGCAGACCCAGGCCTTCTTCGAGGGCTTC |
| | AACGAGATCCTGCCCCAGCAGTACCTGCAGTACTTCGACGCCAAGGAGCTGGAGGTGCTG |
| | CTGTGCGGCATGCAGGAGATCGACCTGAACGACTGGCAGAGGCACGCCATCTACAGGCAC |
| | TACGCCAGGACCAGCAAGCAGATCATGTGGTTCTGGCAGTTCGTGAAGGAGATCGACAAC |
| | GAGAAGAGGATGAGGCTGCTGCAGTTCGTGACCGGCACCTGCAGGCTGCCCGTGGGGGGC |
| | TTCGCCGACCTGATGGGCAGCAACGGCCCCCAGAAGTTCTGCATCGAGAAGGTGGGCAAG |
| | GAGAACTGGCTGCCCAGGAGCCACACCTGCTTCAACAGGCTGGACCTGCCCCCCTACAAG |
| | AGCTACGAGCAGCTGAAGGAGAAGCTGCTGTTCGCCATCGAGGAGACCGAGGGCTTCGGC |
| | CAGGAGTAA |
| | Bold-K393R mutation nucleotides |

In some embodiments, the viral vector is an adeno-associated viral (AAV) vector comprising a polynucleotide sequence encoding an Itch mutant. In some embodiments, the viral vector is selected from AAV2, AAV9, or a combination thereof. In an exemplary embodiment, the AAV vector comprises a combination of AAV2 and AAV9 capsids.

In some embodiments, the viral vector is administered in the form of an injection or an infusion form. In some embodiments, the viral vector is administrated intramuscularly, intravenously, intrathecally, or intracranially in the patient.

In some embodiments, the administration of the viral vector comprising a polynucleotide sequence encoding an Itch mutant to the patient inhibits degradation of TAp73 in neuronal cells of the patient. Accordingly, in some embodiments, provided herein is a method of inhibiting degradation of TAp73 in neuronal cells of a subject comprising administering to the patient a viral vector encoding the Itch mutants described herein. In some embodiments, the viral vector inhibits degradation of TAp73 by about 0.5 to about 4-fold, including values and ranges thereof, compared to levels of TAp73 prior to administration of the viral vector. In some embodiments, the administration of the viral vector inhibits degradation of TAp73 in neuronal cells by about 0.5 to about 3.5-fold, about 0.5 to about 3-fold, about 0.5 to 2.5-fold, about 0.5 to about 2-fold, about 0.5 to about 1.5-fold, about 0.5 to about 1-fold, about 1 to about 4-fold, about 1 to about 3.5-fold, about 1 to about 3-fold, about 1 to about 2.5-fold, about 1 to about 2-fold, about 1 to about 1.5-fold, about 1.5 to about 4-fold, about 1.5 to about 3.5-fold, about 1.5 to about 3-fold, about 1.5 to about 2.5-fold, about 1.5 to about 2-fold, about 2 to about 4-fold, about 2 to about 3.5-fold, about 2 to about 3-fold, about 2 to about 2.5-fold, about 2.5 to about 4-fold, about 2.5 to about 3.5-fold, about 2.5 to about 3-fold, about 3 to about 4-fold, about 3 to about 3.5-fold, or about 3.5 to about 4-fold, including values and ranges thereof, compared to levels of TAp73 prior to administration of the viral vector. In some embodiments, the administration of the viral vector inhibits degradation of TAp73 in neuronal cells by about 0.5-fold, 1-fold, 1.3-fold, 1.4-fold, 1.5-fold, 1.7-fold, 1.8-fold, 2-fold, 2.3-fold, 2.4-fold, 2.5-fold, 2.8-fold, 3-fold, 3.2-fold, 3.4-fold, 3.5-fold, 3.7-fold, or by about 4-fold, compared to levels of TAp73 prior to administration of the viral vector.

In some embodiments, the administration of the viral vector encoding the Itch mutants described herein inhibits degradation of TAp73 in neuronal cells by about 20-80%, including values and ranges thereof, compared to levels of TAp73 prior to administration of the viral vector. In some embodiments, the administration of the viral vector inhibits degradation of TAp73 in neuronal cells by about 20-75%, 20-70%, 20-65%, 20-60%, 20-55%, 20-50%, 20-45%, 20-40%, 20-30%, 25-80%, 25-75%, 25-70%, 25-65%, 25-60%, 25-55%, 25-50%, 25-45%, 25-40%, 25-35%, 30-80%, 30-75%, 30-70%, 30-65%, 30-60%, 30-55%, 30-50%, 30-45%, 35-80%, 35-75%, 35-70%, 35-65%, 35-60%, 35-55%, 35-50%, 40-80%, 40-75%, 40-70%, 40-65%, 40-60%, 40-50%, 50-80%, 50-75%, 50-70%, 50-65%, 50-60%, 60-80%, 60-75%, 60-70%, 65-80%, or 70-80%, including values and ranges thereof, compared to levels of TAp73 prior to administration of the viral vector. In some embodiments, the administration of the viral vector inhibits degradation of TAp73 in neuronal cells by about 20%, 25%, 30%, 35%, 40%, 45%, 50%, 55%, 60%, 65%, 70%, 75%, or about 80%, including values and ranges thereof, compared to levels of TAp73 prior to administration of the viral vector.

In some embodiments, the administration of the viral vector encoding the Itch mutants described here into the patient increases levels of TAp73 in neuronal cells of the patient, compared to levels of TAp73 prior to administration of the viral vector. Accordingly, in some embodiments, provided herein is a method of increasing levels of TAp73 in neuronal cells of a subject comprising administering to the patient a viral vector encoding the Itch mutants described herein. In some embodiments, the administration of the viral vector to the patient increases levels of TAp73 by about 0.5 to about 4-fold, including values and ranges thereof, compared to levels of TAp73 prior to administration of the viral vector. In some embodiments, the administration of the viral vector increases levels of TAp73 in neuronal cells by about 0.5 to about 3.5-fold, about 0.5 to about 3-fold, about 0.5 to 2.5-fold, about 0.5 to about 2-fold, about 0.5 to about 1.5-fold, about 0.5 to about 1-fold, including values and ranges thereof, compared to levels of TAp73 prior to administration of the viral vector. In some embodiments, the administration of the viral vector increases levels of TAp73 in neuronal cells by about 0.5-fold, 1-fold, 1.3-fold, 1.4-fold, 1.5-fold, 1.7-fold, 1.8-fold, 2-fold, 2.3-fold, 2.4-fold, 2.5-fold, 2.8-fold, 3-fold, 3.2-fold, 3.4-fold, 3.5-fold, 3.7-fold, or by about 4-fold, compared to levels of TAp73 prior to administration of the viral vector.

In some embodiments, the administration of the viral vector to the patient increases levels of TAp73 in neuronal cells of the patient by about 20-80%, including values and ranges thereof, compared to levels of TAp73 prior to administration of the viral vector. In some embodiments, the administration of the viral vector increases levels of TAp73 in neuronal cells by about 20-75%, 20-70%, 20-65%, 20-60%, 20-55%, 20-50%, 20-45%, 20-40%, 20-30%, 25-80%, 25-75%, 25-70%, 25-65%, 25-60%, 25-55%, 25-50%, 25-45%, 25-40%, 25-35%, 30-80%, 30-75%, 30-70%, 30-65%, 30-60%, 30-55%, 30-50%, 30-45%, 35-80%, 35-75%, 35-70%, 35-65%, 35-60%, 35-55%, 35-50%, 40-80%, 40-75%, 40-70%, 40-65%, 40-60%, 40-50%, 50-80%, 50-75%, 50-70%, 50-65%, 50-60%, 60-80%, 60-75%, 60-70%, 65-80%, or 70-80%, including values and ranges thereof, compared to levels of TAp73 prior to administration of the viral vector. In some embodiments, the administration of the viral vector increases levels of TAp73 in neuronal cells by about 20%, 25%, 30%, 35%, 40%, 45%, 50%, 55%, 60%, 65%, 70%, 75%, or about 80%, including values and ranges thereof, compared to levels of TAp73 prior to administration of the viral vector.

In some embodiments, the administration of the viral vector encoding the Itch mutants described hereinto the patient inhibits cell cycle related neuronal apoptosis (CRNA) in the patient. Accordingly, in some embodiments, the present disclosure provides a method for inhibiting CRNA in a subject comprising administering to the subject a viral vector encoding the Itch mutants described herein. In some embodiments, the levels of proliferating cell nuclear antigen (PCNA) or the levels of cleaved caspase 3 in neuronal cells of the patient are employed to measure the extent of CRNA.

In some embodiments, the administration of the viral vector inhibits CRNA in the neuronal cells of the patient by about 0.5 to about 5-fold, including values and ranges thereof, as measured by a decrease in levels of PCNA when compared to levels of PCNA prior to administration of the viral vector. That is, in this embodiment, the administration of the viral vector to a patient decreases levels of PCNA by about 0.5 to about 5-fold, including values and ranges thereof, in the neuronal cells of the patient, compared to levels of PCNA prior to administration of the viral vector. In some embodiments, the administration of the viral vector to a patient decreases levels of PCNA by about 0.5 to about 4.5-fold, about 0.5 to about 4-fold, about 0.5 to about 3.5-fold, about 0.5 to about 3-fold, about 0.5 to 2.5-fold, about 0.5 to about 2-fold, about 0.5 to about 1.5-fold, about 0.5 to about 1-fold, about 1 to about 5-fold, about 1 to about 4.5-fold, about 1 to about 4-fold, about 1 to about 3.5-fold, about 1 to about 3-fold, about 1 to about 2.5-fold, about 1 to about 2-fold, about 1 to about 1.5-fold, about 1.5 to about 5-fold, about 1.5 to about 4.5-fold, about 1.5 to about 4-fold, about 1.5 to about 3.5-fold, about 1.5 to about 3-fold, about 1.5 to about 2.5-fold, about 1.5 to about 2-fold, about 2 to about 5-fold, about 2 to about 4.5-fold, about 2 to about 4-fold, about 2 to about 3.5-fold, about 2 to about 3-fold, about 2 to about 2.5-fold, about 2.5 to about 5-fold, about 2.5 to about 4.5-fold, about 2.5 to about 4-fold, about 2.5 to about 3.5-fold, about 2.5 to about 3-fold, about 3 to about 5-fold, about 3 to about 4.5-fold, about 3 to about 4-fold, about 3 to about 3.5-fold, about 3.5 to about 5-fold, about 3.5 to about 4.5-fold, about 3.5 to about 4-fold, or by about 4 to about 5-fold, including values and ranges thereof, compared to levels of PCNA prior to administration of the viral vector. In some embodiments, the administration of the viral vector to a patient decreases levels of PCNA by about 0.5-fold, 1-fold, 1.3-fold, 1.5-fold, 1.8-fold, 2-fold, 2.3-fold, 2.5-fold, 2.8-fold, 3-fold, 3.3-fold, 3.5-fold, 4-fold, 4.3-fold, 4.5-fold, 4.8-fold, or by about 5-fold, compared to levels of PCNA prior to administration of the viral vector.

In some embodiments, the administration of the viral vector to the patient inhibits CRNA in the patient by about 20-90%, including values and ranges thereof, as measured by a decrease in levels of PCNA when compared to levels of PCNA prior to administration of the viral vector. That is, in this embodiment, the administration of the viral vector to a patient decreases levels of PCNA by about 20-90%, including values and ranges thereof, in neuronal cells of the patient, compared to levels of PCNA prior to administration of the viral vector. In some embodiments, the administration of the viral vector to a patient decreases levels of PCNA by about 20-90%, about 20-80%, 20-75%, 20-70%, 20-65%, 20-60%, 20-55%, 20-50%, 20-45%, 20-40%, 20-30%, 25-90%, 25-85%, 25-80%, 25-75%, 25-70%, 25-65%, 25-60%, 25-55%, 25-50%, 25-45%, 25-40%, 25-35%, 30-90%, 30-85%, 30-80%, 30-75%, 30-70%, 30-65%, 30-60%, 30-55%, 30-50%, 30-45%, 35-90%, 35-85%, 35-80%, 35-75%, 35-70%, 35-65%, 35-60%, 35-55%, 35-50%, 40-90%, 40-85%, 40-80%, 40-75%, 40-70%, 40-65%, 40-60%, 40-50%, 50-90%, 50-85%, 50-80%, 50-75%, 50-70%, 50-65%, 50-60%, 60-90%, 60-85%, 60-80%, 60-75%, 60-70%, 65-90%, 65-85%, 65-80%, 70-90%, or 70-80%, including values and ranges thereof, compared to levels of PCNA prior to administration of the viral vector. In some embodiments, the administration of the viral vector to a patient decreases levels of PCNA by about 20%, 25%, 30%, 35%, 40%, 45%, 50%, 55%, 60%, 65%, 70%, 75%, 80%, 85%, or by about 90%, including values and ranges thereof, compared to levels of PCNA prior to administration of the viral vector.

In some embodiments, the administration of the viral vector inhibits CRNA in the neuronal cells of the patient by about 0.5 to about 5-fold, including values and ranges thereof, as measured by a decrease in levels of cleaved caspase-3 when compared to levels of cleaved caspase-3 prior to administration of the viral vector. That is, in this embodiment, the administration of the viral vector to a patient decreases levels of cleaved caspase-3 by about 0.5 to about 5-fold, including values and ranges thereof, in the neuronal cells of the patient, compared to levels of cleaved caspase-3 prior to administration of the viral vector. In some embodiments, the administration of viral vector to a patient decreases levels of cleaved caspase-3 by about 0.5 to about 4.5-fold, about 0.5 to about 4-fold, about 0.5 to about 3.5-fold, about 0.5 to about 3-fold, about 0.5 to 2.5-fold, about 0.5 to about 2-fold, about 0.5 to about 1.5-fold, about 0.5 to about 1-fold, about 1 to about 5-fold, about 1 to about 4.5-fold, about 1 to about 4-fold, about 1 to about 3.5-fold, about 1 to about 3-fold, about 1 to about 2.5-fold, about 1 to about 2-fold, about 1 to about 1.5-fold, about 1.5 to about 5-fold, about 1.5 to about 4.5-fold, about 1.5 to about 4-fold, about 1.5 to about 3.5-fold, about 1.5 to about 3-fold, about 1.5 to about 2.5-fold, about 1.5 to about 2-fold, about 2 to about 5-fold, about 2 to about 4.5-fold, about 2 to about 4-fold, about 2 to about 3.5-fold, about 2 to about 3-fold, about 2 to about 2.5-fold, about 2.5 to about 5-fold, about 2.5 to about 4.5-fold, about 2.5 to about 4-fold, about 2.5 to about 3.5-fold, about 2.5 to about 3-fold, about 3 to about 5-fold, about 3 to about 4.5-fold, about 3 to about 4-fold, about 3 to about 3.5-fold, about 3.5 to about 5-fold, about 3.5 to about 4.5-fold, about 3.5 to about 4-fold, or by about 4 to about 5-fold, including values and ranges thereof, compared to levels of cleaved caspase-3 prior to administration of the viral vector. In some embodiments, the administration of the viral vector to a patient decreases levels of cleaved caspase-3 by about 0.5-fold, 1-fold, 1.3-fold, 1.5-fold, 1.8-fold, 2-fold, 2.3-fold, 2.5-fold, 2.8-fold, 3-fold, 3.3-fold, 3.5-fold, 4-fold, 4.3-fold, 4.5-fold, 4.8-fold, or by about 5-fold, compared to levels of cleaved caspase-3 prior to administration of the viral vector.

In some embodiments, the administration of the viral vector to the patient inhibits CRNA in the patient by about 20-90%, including values and ranges thereof, as measured by a decrease in levels of cleaved caspase-3 when compared to levels of cleaved caspase-3 prior to administration of the viral vector. That is, in this embodiment, the administration of the viral vector to a patient decreases levels of cleaved caspase-3 by about 20-90%, including values and ranges thereof, in neuronal cells of the patient, compared to levels of cleaved caspase-3 prior to administration of the viral vector. In some embodiments, the administration of the viral vector to a patient decreases levels of cleaved caspase-3 by about 20-90%, about 20-80%, 20-75%, 20-70%, 20-65%, 20-60%, 20-55%, 20-50%, 20-45%, 20-40%, 20-30%, 25-90%, 25-85%, 25-80%, 25-75%, 25-70%, 25-65%, 25-60%, 25-55%, 25-50%, 25-45%, 25-40%, 25-35%, 30-90%, 30-85%, 30-80%, 30-75%, 30-70%, 30-65%, 30-60%, 30-55%, 30-50%, 30-45%, 35-90%, 35-85%, 35-80%, 35-75%, 35-70%, 35-65%, 35-60%, 35-55%, 35-50%, 40-90%, 40-85%, 40-80%, 40-75%, 40-70%, 40-65%, 40-60%, 40-50%, 50-90%, 50-85%, 50-80%, 50-75%, 50-70%, 50-65%, 50-60%, 60-90%, 60-85%, 60-80%, 60-75%, 60-70%, 65-90%, 65-85%, 65-80%, 70-90%, or 70-80%, including values and ranges thereof, compared to levels of cleaved caspase-3 prior to administration of the viral vector. In some embodiments, the administration of the viral vector to a patient decreases levels of cleaved caspase-3 by about 20%, 25%, 30%, 35%, 40%, 45%, 50%, 55%, 60%, 65%, 70%, 75%, 80%, 85%, or by about 90%, including values and ranges thereof, compared to levels of cleaved caspase-3 prior to administration of the viral vector.

The administration of the viral vector encoding the Itch mutants described herein to a subject reduces the rate of neurodegeneration in the subject as measured by a decrease in the levels of PCNA or cleaved caspase-3 in the neuronal cells of the subject or by an increase in the levels of TAp73 in the neuronal cells of the subject. Accordingly, provided herein is a method of reducing neurodegeneration in a patient suffering from cognitive impairment or at a risk of developing AD, comprising administering to the patient a viral vector encoding the Itch mutants described herein. The rate or the extent of neurodegeneration can be measured by measuring the levels of TAp73, PCNA, cleaved caspase-3 in a sample obtained from the subject.

In some embodiments, the administration of the viral vector encoding the Itch mutants described herein improves the cognitive function, learning abilities, and/or memory of the patients. In some embodiments, the administration of the viral vector improves a long-term and short-term memory in the patients.

In some embodiments of the present invention provides a composition comprising a viral vector comprising a polynucleotide sequence encoding an Itch mutant for use in treating a cognitive impairment due to Itch dysregulation.

In some embodiments, the Itch mutant expressed by the viral vector comprises a mutation selected from the group consisting of: T222A, S232A, K393R and a combination thereof. In some embodiments, the Itch mutant expressed by the viral vector comprises a mutation selected from the group consisting of: T222A, S232A, K391R and a combination thereof.

In some embodiments, the Itch mutant expressed by the viral vector comprises both T222A and S232A mutations. In some embodiments, the Itch mutant comprises T222A or S232A mutation.

In some embodiments, the Itch mutant expressed by the viral vector comprises K393R mutation. In some embodiments, the Itch mutant expressed by the viral vector comprises K391R mutation.

In some embodiments, the viral vector of the composition is an Adeno-Associated Viral (AAV) vector.

In some embodiments, the AAV vector of the composition is AAV2 or AAV9 vector or a combination thereof. In an exemplary embodiment, the AAV vector comprises a combination of AAV2 and AAV9 capsids.

In some embodiments, the composition is in an injection or an infusion form.

In some embodiments, the composition inhibits degradation of TAp73 in neuronal cells of a patient by about 0.5 to about 2-fold compared to levels of TAp73 prior to administration of the composition.

In some embodiments, the composition inhibits degradation of TAp73 in neuronal cells of a patient by about 20-70% compared to levels of TAp73 prior to administration of the composition.

In some embodiments, the composition inhibits cell cycle related neuronal apoptosis (CRNA) in a patient by about 0.5 to 5-fold as measured by a decrease in levels of proliferating cell nuclear antigen (PCNA) when compared to levels of PCNA prior to administration of the composition.

In some embodiments, the composition inhibits cell cycle related neuronal apoptosis (CRNA) in the patient by about 20-90% as measured by a decrease in levels of proliferating cell nuclear antigen (PCNA) when compared to levels of PCNA prior to administration of the composition.

In some embodiments, the composition improves short-term and long-term memory in a patient.

In some embodiments, the composition improves the learning ability of the patient.

In some embodiments, the composition is used for the treatment of the cognitive impairment due to Itch dysregulation associated with a degenerative neurological disorder.

In some embodiments, the composition is used for the treatment of the degenerative neurological disorder such as Alzheimer's disease.

Alzheimer's disease has a spectrum of clinical stages depending on the severity of cognitive and/or functional impairment. Some of the clinical stages of AD include, but are not limited to, a pre-clinical stage, mild cognitive impairment (MCI), mild AD, moderate AD, and an advanced AD. In some embodiments, the present disclosure provides a method for treating a subject having a pre-clinical stage of AD, comprising administering to the subject a viral vector comprising a polynucleotide sequence encoding the Itch mutants described herein. In some embodiments, the present disclosure provides a method for treating a subject having a mild cognitive impairment (MCI), comprising administering to the subject said viral vector.

In some embodiments, the present disclosure provides use of a viral vector comprising a polynucleotide sequence encoding the Itch mutants described herein for treating cognitive impairment. AD is a progressive disease related to the nervous system and can be categorized into various stages based on the severity of symptoms as described herein. The present disclosure contemplates use of a viral vector comprising a polynucleotide sequence encoding an Itch mutant for treating various stages of AD described herein. Dosage forms, and routes of administration of the viral vector are discussed above.

In some embodiments, the present disclosure provides a viral vector comprising a polynucleotide sequence encoding the Itch mutants described for use as a medicament for treating various stages of cognitive impairment due to Itch dysregulation. Dosage forms and routes of administration that may be employed for the medicament are discussed above.

The recombinant AAV vectors comprising the polynucleotide of interest of the present disclosure may be produced using any convenient methodologies, AAV packaging cells, and packaging technology as known to those of skill in the art. For example, an AAV expression vector (that is, a plasmid comprising the rAAV genome as well as elements useful for the cloning of the genomic elements in, e.g., bacteria, e.g., origin of replication, selectable marker, etc.) may be transfected into mammalian producer cells. Also transfected into the mammalian producer cells is an AAV helper construct, i.e., a plasmid comprising AAV REP and CAP coding regions that can be expressed in the producer cell, which complement AAV helper functions absent from the AAV expression vector. The dually transfected producer cells are then infected by a helper virus, e.g. adenovirus, or transfected with a plasmid comprising helper virus accessory genes that promote AAV vector replication, e.g., regions VA, E2A, E4, so as to promote efficient rAAV virus production. The producer cells are then cultured to produce rAAV, and AAV vectors are purified and formulated using standard techniques known in the art.

In some embodiments, the rAAV are of a wild-type serotype; that is, they comprise a viral capsid that consists of viral capsid proteins that occur in nature. In other embodiments, the rAAV are an AAV serotype variant, i.e., they comprise a variant AAV capsid protein, that is, an AAV capsid protein that comprises at least one amino acid difference relative to a corresponding parental AAV capsid protein, e.g., a wild type AAV capsid protein, and does not consist of an amino acid sequence present in a naturally occurring AAV capsid protein. In some embodiments, the rAAV vector is AAV2 or AAV9 serotype.

To express a polynucleotide encoding an Itch mutant, the polynucleotide is operably linked to a promoter. In some embodiments, the viral genome comprising the polynucleotide encoding an Itch mutant also comprises a promoter, wherein the promoter is operably linked to the polynucleotide to form an expression cassette. In some other embodiments, the polynucleotide encoding an Itch mutant is flanked by sequences that promote the integration of the polynucleotide into the host cell, i.e., a neuronal cell. In other words, the viral genome comprising the polynucleotide encoding an Itch mutant comprises sequences flanking the polynucleotide that are homologous to sequences flanking the 3′ end of a host cell promoter and promote the recombination of the polynucleotide into the host genome such that it is operably linked to the host cell promoter.

In some embodiments where the rAAV comprises a promoter operably linked to the polynucleotide encoding an Itch mutant, the promoter is a ubiquitous promoter, i.e., it is a promoter that is active in a wide range of cells, tissues and species. In other instances, the promoter is a neuronal cell specific promoter. Non-limiting examples of neuronal cell specific promoters that can be employed in the present vectors include neuronal-specific enolase (NSE) promoter, glial fibrillary acidic protein (GFAP) promoter, human synaps in (hSyn) promoter, phosphoglycerate kinase (PGK) promoter, polyubiquitin C (UbC) promoter and modified chicken beta-actin (CBh) promoter.

It is to be understood that the foregoing descriptive matter is illustrative of the disclosure and not a limitation. While considerable emphasis has been placed herein on the particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1

### Methods and Materials

### (i) Preparation of Primary Cell Culture and Treatments

Cortical neurons from embryonic day 18 (E18) Sprague-Dawley rats or Embryonic day 16 (E16) APP/PS1 transgenic AD mice were isolated and cultured as previously published (Modi, Komaravelli et al. 2012, Modi, Jaiswal et al. 2016, Chauhan, Modi et al. 2020). Time mating for animals were setup and 16-18 days pregnant were sacrificed thereafter, E18 rat or E16 mouse embryos were dissected, and cortical region of the brain was isolated and trypsinized followed by addition of Serum Containing (10% fetal bovine serum and 10% horse serum) Media (SCM). Cell pellet was resuspended in SCM and plated on poly-L-Lysine coated 6-well plates. After 12 h, cells were washed with Tyrode's CMF PBS supplemented with glucose and NaHCO₃ and were maintained in Serum Free Medium (SFM) containing B27 and N2 supplement (Gibco, Life technologies), 1× penicillin-streptomycin, L-glutamine, and glucose in a CO₂ incubator (5% CO₂ levels), for five days. Typically, in vitro transfections or Aβ₁₋₄₂ treatments were performed at fifth day in vitro (DIV).

HEK293T/A (Human Embryonic Kidney) cells were maintained in DMEM with 10% fetal bovine serum and 1× antibiotic/antimycotic at 37° C. in 5% CO₂. Most of the treatments were typically done for 48 h. 0.5 μM of soluble oligomers of Aβ₁₋₄₂ (R-peptide) was used as described previously (Stine, Dahlgren et al. 2003, Modi, Komaravelli et al. 2012, Modi, Jaiswal et al. 2016, Chauhan, Modi et al. 2020) for 48 h.

### Western Blotting:

Cells were washed with 1×PBS and lysed using ice cold complete lysis buffer (100 mM Tris-HCl pH 7.4, 5 mM EDTA, 100 mM NaCl, 1% Triton ×100 and 10% Glycerol, 1 mM phenyl methane sulfonyl fluoride (PMSF), 1 mM sodium orthovanadate, 20 mM β-glycero-phosphate and 1× protease inhibitor cocktail was added before use). Immunoblotting was performed by using primary antibodies and secondary antibody conjugated with horse radish peroxidase (HRP). Chemiluminescence reagent West Pico or West Dura (Pierce) was used for detection as per manufacturer's instructions.

For lysate preparations from tissues, mice were deeply anesthetized and perfused with ice-cold 1×PBS. Cortex and hippocampus were dissected out from perfused brain and stored at −80° C. for immunoblotting analysis or mRNA expression analysis. For immunoblotting, equal amount of tissue from each mouse was minced in RIPA lysis buffer (100 mM Tris-HCl pH 7.4, 50 mM NaCl, 50 mM EDTA and 1% Triton X-100 and supplemented with a protease inhibitor cocktail (Roche), 2% SDS and 1 mM phenyl methane sulfonyl fluoride) and homogenized. Protein was estimated with BCA solutions (G-biosciences).

### (ii) Construction of Itch and its Mutants AAV Preparation and Characterization:

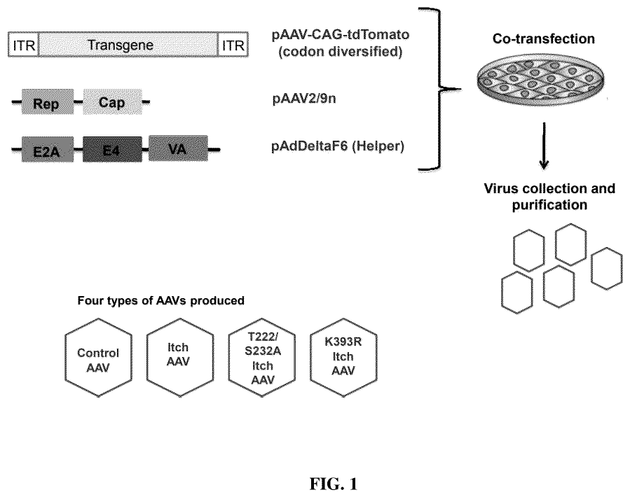

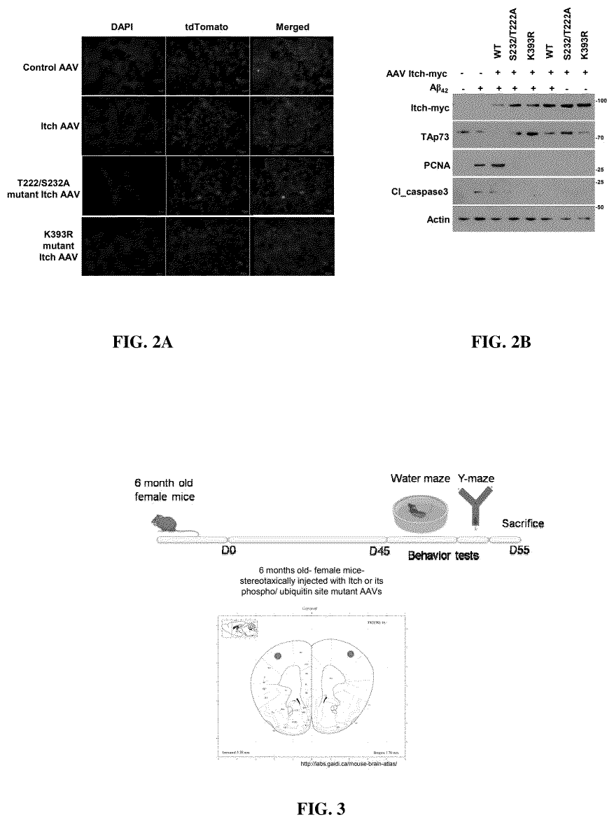

Mus musculus Itch protein coding DNA sequence was cloned in the AAV expression vector. Different expression construct having mutations in Proline rich region Threonine 222/Serine 232 to Alanine (TS/A) and WW domain, Lysine 393 to Arginine (K/R) were created with site directed mutagenesis in TA-vectors and then sub-cloned into AAV expression vector. Viral particles were prepared by co-transfecting this construct along with AAV packaging plasmids in HEK293T cells which then harvested (FIG. 1) and concentrated. The viability of AAV prepared was measured by the Td-Tomato fluorescence after transducing in HEK293T cells (FIG. 2A). These AAVs were also transduced in rat primary cortical neurons. While, all the Itch (WT/mutant) AAVs were expressing in neurons (FIG. 2B lane 3 to 8), the Itch mutants were also able to restore the TAp73 protein levels which were downregulated with Aβ42 treatment (FIG. 2B) thereby preventing CRNA.

Recombinant AAV was created by cloning mouse Itch protein coding sequence in pAAV-CAG-tdTomato vector (pAAV-CAG-tdTomato (codon diversified) was obtained from Edward Boyden (Addgene plasmid #59462; http://addgene.org/59462; RRID: Addgene_59462). Briefly, Itch cDNA along with myc-tag was PCR amplified from pCIneo-myc-Itch (Addgene plasmid #2859) with EcoRI restriction enzyme sites on both ends. Vector and insert both were digested separately with EcoRI restriction enzyme, purified and ligated. Positive clones and orientation of Itch CDS was confirmed by multiple enzyme restriction digestions. Similarly, the TS/A (T222 and S232 to A) and K/R (K393 to R) mutation were introduced in the plasmid with site directed mutagenesis. HEK293T cells were co-transfected with cis-plasmid pAAV-tdTomato or pAAV-Itch or pAAV-TS/A Itch or pAAV-K/R Itch with trans-plasmid pAAV2/9n (obtained from James M. Wilson; Addgene #112865; http://addgene.org/112865; RRID: Addgene_112865) and a helper plasmid pAdDeltaF6 (obtained from James M. Wilson; Addgene #112867; http://addgene.org/112867; RRID: Addgene_112867) to produce AAV. Cells were harvested after 72 h, cells were lysed using freeze-thaw method and virus particles were concentrated and resuspended in AAV storage buffer (1×PBS, 35 mM sodium chloride and 5% glycerol) (Lock, Alvira et al. 2010, Wu, Huang et al. 2012). For number of transducing unit (TU/ml) determination, rat cortical neurons were seeded at a density of 0.5*106 cells/well on cover slips in a 24-well plate. They were transduced with AAVs in 200 μl serum free media then 300 μl fresh media were added 24 hours after transduction. A half volume of media was exchanged with fresh media on the following day and every 2-3 days. Transducing units were calculated by counting the RFP positive cells out of total number of cells on coverslip.

(iii) Expression of Itch Mutants

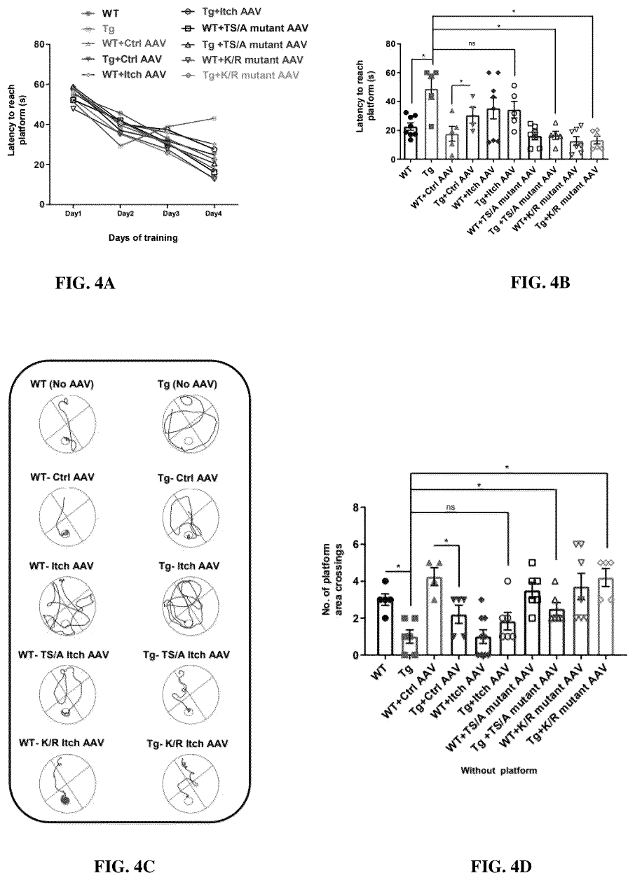

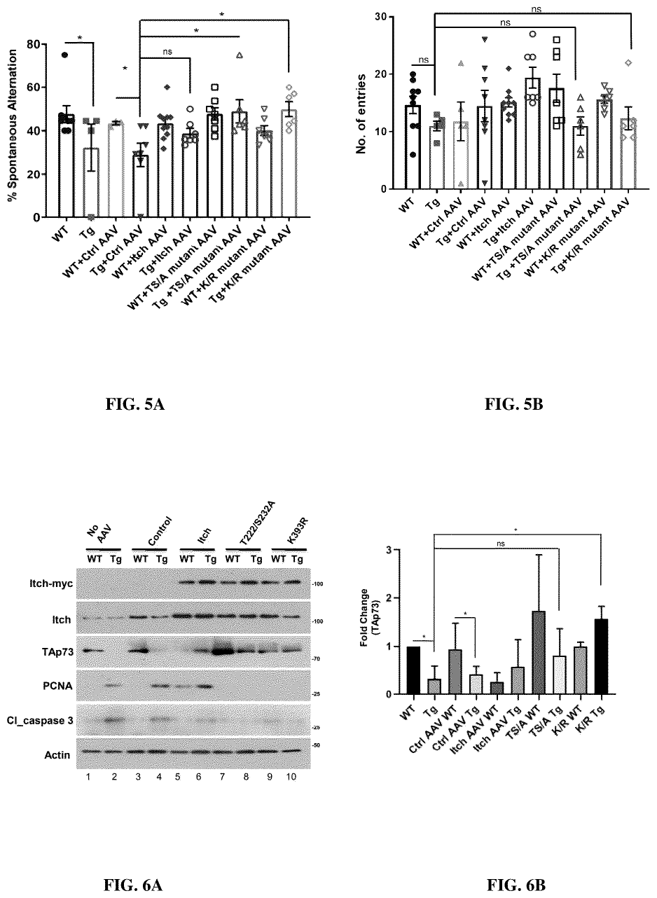

The AAV (serotype 2/9) were stereotaxically injected in the frontal cortex region of WT and TgAD mice at 6 months of age. AAV-tdTomato at the same dose was injected as a control. After 45 days of AAV delivery, behavioral tests were performed (FIG. 3). As expected, compared with WT littermates, untreated TgAD mice showed significant cognitive decline observed with Morris water maze and Y-maze tests analysis (FIGS. 4A-4D and 5A-5B respectively). AAV-tdTomato-treated TgAD mice had similar behavioral performances to untreated TgAD mice (FIGS. 4A-4D and 5A-5B), indicating that the injection of AAV-tdTomato and expression of modified RFP had no effect on the cognition of these mice.

As shown in FIGS. 4A and 4B, in the Morris water maze test, Tg-Itch-AAV mice showed no change in the latency to reach hidden platform even at end of training, while the latencies of Tg-AAV-TS/A Itch or Tg-AAV-K/R Itch decreased significantly and were almost similar to the WT mice. These findings were also evident while looking at the track plots of some the representative mice in FIG. 4C.

The probe test (without) displayed the similar results, as Tg-AAV-Itch mice showed no change in the number of platform area crossings (FIG. 4D) while Tg-AAV-TS/A Itch or Tg-AAV-K/R Itch crossed the platform area significantly higher than the TgAD mice (FIG. 4D). There was no significant difference in the swim speed, food or water intake among all the experimental and control groups.

In comparison to untreated or AAV-tdTomato TgAD mice, the TgAD or WT mice treated with AAV-Itch performed worse in the Y-maze, as reflected by a significant decrease in the spontaneous alternation percentage while the TgAD mice injected with Itch mutants showed significant increase in the spontaneous alternation percentage (FIG. 5A) with no significant changes in total entry number (FIG. 5B) in the spontaneous alternation test. These results indicate that the Itch mutants are protecting the TgAD mice from cognitive decline.

### Results

### Example-2: Viral Vector Prevents TAp73 Degradation and Reverts CRNA

After all the behavioral tests, these mice were sacrificed, and brain tissues were prepared for various biochemical analysis. The western blot analysis (FIGS. 6A and 6B) of brain tissue isolated from these injected mice revealed the myc-tag expression in case of Itch-AAV, T222/S232A and K393R injected animals.

The immunoblotting from the cortex tissue lysates revealed that TAp73 was found to be downregulated in TgAD or control AAV injected TgAD neurons (FIG. 6A lane 2 and 4 vs 1 and 3 respectively), which was rescued with the expression of AAV-T222/S232A (FIG. 6A lane 8) or AAV-K393R Itch (FIG. 6A lane 10) in TgAD cortex while AAV-Itch injection made TAp73 expression even worse in WT animals.

As mentioned earlier TAp73 plays a crucial role in controlling the cell cycle of terminally differentiated neurons, the TgAD cortices had increased amounts of PCNA and cleaved caspase 3 as compared to the WT cortex (FIG. 6A lane 2 and 4 vs 1 and 3 respectively). In correlation with the TAp73 levels, the mice injected with AAV-T222/S232A or K393R Itch showed a very less or no PCNA or cleaved caspase 3 levels while WT Itch overexpression caused a significant increase in the PCNA and cleaved caspase 3 levels even in the WT mice cortex (FIG. 6A lane 5 and 6). These data suggest that Itch AAV mutant expression is able to rescue TAp73 degradation and protect neurons from cell cycle re-entry and apoptosis.

### Example 3: Viral Vector-Treated Mice Exhibit Reduced CRNA and Improves Cognition Defects

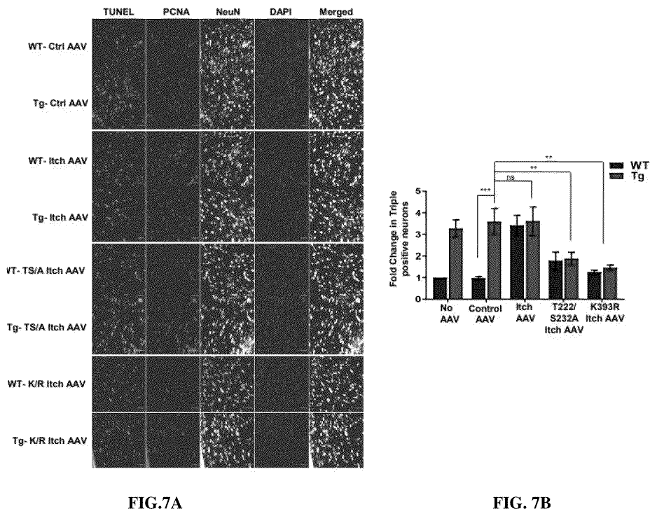

Immunohistochemistry for cell cycle reactivation and apoptosis was also performed. Immunostaining for PCNA in combination TUNEL labeling on frontal cortex cryosections to identify the neurons undergoing cell cycle re-entry (PCNA+) and also apoptosis (TUNEL+) were identified. In addition, anti-NeuN antibody was used to specifically label the neurons. Previously, aberrant cell cycle re-entry related neurodegeneration has been reported in the cortex as well as the hippocampus of various AD animal models (Busser, Geldmacher et al. 1998, Yang, Mufson et al. 2003, Varvel, Bhaskar et al. 2008). The TgAD mice cortex revealed significantly higher TUNEL⁺/PCNA⁻, TUNEL⁻/PCNA⁺ (FIG. 7A), as well as PCNA+/TUNEL+ neurons in comparison to WT mice (FIG. 7A). Similar observations were made in control AAV injected TgAD cortex. In sharp contrast, AAV-Itch injection resulted in a huge increase in TUNEL+/PCNA+WT neurons as well; while the AAV-T222/S232A Itch or AAV-K393R Itch TgAD injections showed a significant decrease in the number of triple positive neurons (FIG. 7B). These data confirmed that Itch abundance/activation can contribute to CRNA in TgAD mice, which can be overcome by overexpression of its mutants T222/S232A or K393R mutant in the cortex of these animals. Importantly, these results support and provide and the mechanism for the reversal of defects in learning and memory in TgAD mice upon microinjection of AAVs expressing these mutant Itch copies.

### Behavioural Tests:

For Morris water maze test, animals were subjected to three trials per session and one session a day, for five days. Each trial was for 60 s in which mice had to reach the hidden platform in the water, and the average time per session was recorded as escape latency. After the escape training, the platform was removed for the probe trial test. It tested the retention of spatial memory at 24 h after training (Vorhees and Williams 2006, Nunez 2008). The number of times a mouse crossed the probe, the time spent in the target quadrant, and swim speed were recorded using ANY-maze software (Stoelting Co., USA).

For Y-Maze test, animals were habituated in the Y-shaped maze for 3 min with one of the arms blocked then their spontaneous alternations were recorded with all arms open for 5 min (Miedel, Patton et al. 2017). The number of alternations was recorded manually, and percent spontaneous alternations were calculated as mentioned below:

Percentage spontaneous alternation (SA)=(No. of alternations/(No. of arm entries−2)*100

For radial arm (8-arm) maze test, mice were given restricted amount of food until their body weight reduced to 85% of the initial weight for two days prior to habituation in the maze. In habituation phase, each mouse was allowed to explore and consume food pellets scattered on the whole maze for a 10 min period (one session per mouse). In the training phase, three arms were blocked and the remaining five alternate arms were baited. On the test day, all arms were opened and same five arms were baited as in trial sessions and their series of entries were recorded manually (Miyakawa, Yamada et al. 2001), from which the percentage of working and reference memory errors were calculated. The mouse entering an arm containing food where it had previously entered, was counted as working memory error (short term memory) and if the mouse entered in an arm without bait, was counted as reference memory error (long term memory for position of the baited arms).

### Immunohistochemistry (IHC) and TUNEL Labelling on Brain Cryosections

After performing behavioral tests on stereotaxically injected animals, animals were deeply anesthetized and perfused with 1×PBS followed by 4% PFA. Brains were isolated and kept in 4% PFA for 12 h and then in 30% sucrose until the brains sink to the bottom of tube. Blocks were prepared using Leica Tissue freezing medium and stored at −80° C. 10 μm sections on poly-L-Lysine coated glass slides were prepared on a Leica cryotome.

For TUNEL labelling and IHC, slides with brain sections were twice washed with 1×PBS for 10 min at room temperature. Next, heat-mediated citrate buffer antigen retrieval (AR) was performed by boiling for 10 min and gradual cooling at room temperature for 20 min and then rinsed with distilled water (Moreno-Jimenez, Flor-Garcia et al. 2019). After 1×PBS wash, proteinase K treatment (5 min) was given followed by 4% PFA fixation for 3 min followed by TUNEL labeling performed by using Dead End fluorometric TUNEL system (G3250, Promega) as per manufacturer's guidelines. Subsequently, blocking was done with 3% BSA, 2% normal goat serum and 0.15% Triton X 100 for 1 h followed by incubation with primary antibodies at 4° C. for 12 h. To detect binding of primary antibodies, the Alexa Fluor (488 or 594) conjugated or r-PE conjugated secondary antibodies were used. Sections were mounted with DAPI containing medium (VectaShield) Single-plane four-channel (DAPI (blue), TUNEL (green), PCNA and NeuN (yellow)) 8-bit images (25× oil-immersion objective) were obtained with Zeiss AxioImager1 microscope. The acquisition settings were kept constant for all images. Five fields per section and three sections per mouse were obtained for quantification of triple positive cells.

### Real-Time Quantitative PCR

About 50 mg region-matched brain tissue from 6-month-old WT and TgAD (treated/untreated) mice were homogenized in 1 ml TRIZOL reagent (15596-018, Invitrogen) and incubated for 5 min at room temperature. Tissue extracts were centrifuged at 4° C., 12,000×g for 5 min. 500 ml supernatant was used for RNA extraction by Trizol reagent, according to the manufacturer's instruction. RNA concentration was measured and reverse transcription of total RNA to complementary DNA was performed using M-MuLV Reverse Transcriptase (M1705, Promega). Quantitative PCR was performed using the CFX95 BioRad real time PCR System. PCR products were detected by SYBR Premix (F416L, Thermo).

For in vivo studies, ~Further in vivo studies were carried out, in which these mutants expressing adeno-associated virus (AAV2/9n) were stereotaxically injected into TgAD mouse brain (bilateral injections) and the mice were subjected to memory and learning behaviour tests. Amyloid Precursor Protein (APP)/Presenilin 1 (PS1) transgenic (TgAD) mouse model for AD (strain name B6C3-Tg APPs we, PSEN1dE9 85Dbo/J; stock number 004462) maintained at the Jackson laboratory was obtained from National Brain Research Centre, Manesar to NII, New Delhi. TgAD mouse model expresses a chimeric mouse/human amyloid-β precursor protein containing two familial Swedish mutations (K595N and M596L) and a mutant human presenilin1 gene carrying the exon-9 deletion under mouse prion promoter elements, directing transgene expression predominantly to the central nervous system neurons. The levels of Aβ42 produced are significantly higher in female animals (Jankowsky, Slunt et al. 2001, Jankowsky, Fadale et al. 2004). Wild type and transgenic (TgAD) mice were genotyped using genomic DNA isolated from mouse tail and PCR primers according to the Jackson's laboratory protocol.

The mouse injected with K393R Itch or T222-S232/A Itch AAV showed an improvement in the cognition as compared to the control AAV injected TgAD mouse. These and other related findings suggest that Itch is a therapeutic target for Alzheimer's disease and AAV expressing the above-motioned mutants Itch can be used for this purpose.

1. A method for treating a cognitive impairment due to Itch dysregulation in a patient in need thereof, comprising administering to the patient a viral vector comprising a polynucleotide sequence encoding an Itch mutant.

2. The method of claim 1, wherein the Itch mutant comprises a mutation selected from the group consisting of: T222A, S232A, K391R and a combination thereof.

3. The method of claim 1, wherein the Itch mutant comprises T222A and S232A mutations.

4. The method of claim 1, wherein the Itch mutant comprises K391R mutation.

5. The method of claim 1, wherein the viral vector is administered intramuscularly, intravenously, or intrathecally.

6. The method of claim 1, wherein the viral vector is adeno-associated viral (AAV) vector.

7. The method of claim 1, wherein the AAV vector comprises an AAV2 capsid, an AAV9 capsid, or a combination of AAV2 and AAV9 capsids.

8. The method of claim 1, wherein the administration of the viral vector inhibits degradation of TAp73 in neuronal cells of the patient.

9. The method of claim 1, wherein administration of the viral vector to the patient inhibits degradation of TAp73 in neuronal cells of the patient by about 0.5 to about 2-fold compared to levels of TAp73 prior to administration of the viral vector.

10. The method of claim 1, wherein administration of the viral vector to the patient inhibits degradation of TAp73 in neuronal cells of the patient by about 20-70% compared to levels of TAp73 prior to administration of the viral vector.

11. The method of claim 1, wherein administration of the viral vector to the patient inhibits cell cycle related neuronal apoptosis (CRNA) in the patient by about 0.5 to 5-fold as measured by a decrease in levels of proliferating cell nuclear antigen (PCNA) when compared to levels of PCNA prior to administration of the viral vector.

12. The method of claim 1, wherein administration of the viral vector to the patient inhibits cell cycle related neuronal apoptosis (CRNA) in the patient by about 20-90% as measured by a decrease in levels of proliferating cell nuclear antigen (PCNA) when compared to levels of PCNA prior to administration of the viral vector.

13. The method of claim 1, wherein administration of the viral vector to the patient improves short-term and long-term memory in the patient.

14. The method of claim 1, wherein administration of the viral vector to the patient improves a learning ability of the patient.

15. The method of claim 1, wherein the cognitive impairment due to Itch dysregulation is associated with a degenerative neurological disorder.

16. The method of claim 1, wherein the degenerative neurological disorder is Alzheimer's disease.

17.-38. (canceled)

39. A recombinant viral vector comprising a polynucleotide sequence encoding an Itch mutant operably linked to a promoter.

40. The recombinant viral vector of claim 39, wherein the polynucleotide sequence encodes an Itch mutant comprising T222A and S232A mutations.

41. The recombinant viral vector of claim 39, wherein the polynucleotide sequence encodes an Itch mutant comprising K391R mutation.

42. The recombinant vector of claim 39, wherein the vector is an adeno-associated viral (AAV) vector.


SEQUENCE LISTING (20 sequences)

SEQ ID NO: 1 - DNA, 8775 bp, synthetic construct
  source 1..8775: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggtgatggagcagaagctgatttccgaggaggacctgaacggtggtagtctgaccatgaaatctcaacttcagatcactgtcatctcagccaaacttaaagaaaataaaaagaattggtttggaccaagtccttatgtagaagtcacagtagatggacagtcaaagaagacagaaaaatgcaataatacaaacagtcccaagtggaagcagcccctcacagttattgttacccctacgagtaaattatgttttcgtgtgtggagtcaccagaccctgaagtctgatgttttattgggaactgctggattagatatttatgaaacattaaagtcaaacaatatgaaacttgaagaagtagttatgactttgcagcttgtaggtgacaaagagccaacagagacgatgggagatttgtcagtttgtcttgatgggctgcaagtagaagctgaggttgttactaacggtgaaacgtcatgctccgagagtactacacagaatgatgatggctgcagaaccagagatgatacaagagtgagcacaaatggatcagaggaccctgaggttgcagcgtcaggggaaaacaagagggccaatgggaacaattctccgtccctttcaaatggtggttttaagccttctagacctcctagaccttcgagaccacctccacccaccccacgaagaccagcttctgtcaatggctcaccatccacgaattctgacagtgatggatctagtacaggctccttgccaccaacaaatacaaatgtaaatacaagtacatctgaaggagcaacatctggattaataattcctcttactatatctggaggctcgggccctaggcctctgaatactgtaagccaagctcccctaccacctgggtgggagcagagagtagaccagcatgggcgtgtttactatgtagaccatgttgaaaagcgaacaacatgggatagaccagaacctctacctcctggctgggaacggcgtgtggacaatatgggacgtatttattatgttgatcatttcacaagaaccacaacatggcagaggccaaccttggaatctgtccggaactatgaacagtggcagctacagcgtagtcagcttcagggagcaatgcagcagtttaaccagagattcatctatgggaatcaagatttgtttgctacatcacaaaacaaagaatttgatccgcttggcccgttgccccctggatgggagaagagaactgatagcaatggcagagtgtattttgtcaaccacaacactcggattactcagtgggaagaccccagaagccaaggtcagttaaatgaaaagcccttaccagaaggctgggaaatgagattcacagtggatggaattccatattttgtggaccacaatagaagagcaactacttatatagatccacgaacgggaaaatcagccttagacaatgggccccagatagcctatgtgcgggacttcaaggcaaaagttcagtatttccggttctggtgccagcaactggccatgccacagcatataaagatcacagtgacaagaaaaacattgtttgaggattcctttcagcagatcatgagcttcagcccgcaagacctgagaagacgtttgtgggtgattttcccaggagaagaaggtttagattatggaggtgtagcaagagaatggttctttcttttgtcacatgaagtgttgaacccaatgtattgcctgtttgaatatgcagggaaggataactactgcctgcagataaaccccgcttcttacatcaatccagaccacctgaaatactttcgttttattggcagatttattgccatggctctgtttcatgggaaattcatagataccggattttctttaccattctataaacgtatcctgaataaaccagttggacttaaggatttagaatctattgatccagaattttataattcgctcatctgggttaaagaaaacaacatcgaagaatgtggtttggaaatgtacttctcagttgataaagaaattctaggtgaaattaagagtcatgatttgaaaccgaatggtggcaatattcttgtgacagaagaaaacaaggaggaatacatcaggatggtagctgagtggaggttgtctcgaggtgttgaagaacagacacaagctttctttgagggctttaatgaaattcttcctcagcaatatttgcaatattttgatgcgaaggaattagaggttcttctgtgtgggatgcaggagattgatttgaatgactggcagagacatgccatctaccgccactacaccagaacaagcaagcagatcatgtggttttggcagtttgttaaagaaattgataatgagaagaggatgagacttctgcagtttgttactggaacctgccgattgccagtgggaggatttgctgaccttatggggagtaatggaccacagaagttctgcatcgaaaaagttggcaaagaaaattggttacccagaagccatacttgttttaaccgcctggacctcccaccttacaagagctatgagcaactgaaggaaaagctgttatttgccattgaagaaactgaaggatttggacaagagggatccggtaccgccaccatggtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgct

SEQ ID NO: 2 - DNA, 8775 bp, synthetic construct
  source 1..8775: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggtgatggagcagaagctgatttccgaggaggacctgaacggtggtagtctgaccatgaaatctcaacttcagatcactgtcatctcagccaaacttaaagaaaataaaaagaattggtttggaccaagtccttatgtagaagtcacagtagatggacagtcaaagaagacagaaaaatgcaataatacaaacagtcccaagtggaagcagcccctcacagttattgttacccctacgagtaaattatgttttcgtgtgtggagtcaccagaccctgaagtctgatgttttattgggaactgctggattagatatttatgaaacattaaagtcaaacaatatgaaacttgaagaagtagttatgactttgcagcttgtaggtgacaaagagccaacagagacgatgggagatttgtcagtttgtcttgatgggctgcaagtagaagctgaggttgttactaacggtgaaacgtcatgctccgagagtactacacagaatgatgatggctgcagaaccagagatgatacaagagtgagcacaaatggatcagaggaccctgaggttgcagcgtcaggggaaaacaagagggccaatgggaacaattctccgtccctttcaaatggtggttttaagccttctagacctcctagaccttcgagaccacctccacccgctccacgaagaccagcttctgtcaatggcgcaccatccacgaattctgacagtgatggatctagtacaggctccttgccaccaacaaatacaaatgtaaatacaagtacatctgaaggagcaacatctggattaataattcctcttactatatctggaggctcgggccctaggcctctgaatactgtaagccaagctcccctaccacctgggtgggagcagagagtagaccagcatgggcgtgtttactatgtagaccatgttgaaaagcgaacaacatgggatagaccagaacctctacctcctggctgggaacggcgtgtggacaatatgggacgtatttattatgttgatcatttcacaagaaccacaacatggcagaggccaaccttggaatctgtccggaactatgaacagtggcagctacagcgtagtcagcttcagggagcaatgcagcagtttaaccagagattcatctatgggaatcaagatttgtttgctacatcacaaaacaaagaatttgatccgcttggcccgttgccccctggatgggagaagagaactgatagcaatggcagagtgtattttgtcaaccacaacactcggattactcagtgggaagaccccagaagccaaggtcagttaaatgaaaagcccttaccagaaggctgggaaatgagattcacagtggatggaattccatattttgtggaccacaatagaagagcaactacttatatagatccacgaacgggaaaatcagccttagacaatgggccccagatagcctatgtgcgggacttcaaggcaaaagttcagtatttccggttctggtgccagcaactggccatgccacagcatataaagatcacagtgacaagaaaaacattgtttgaggattcctttcagcagatcatgagcttcagcccgcaagacctgagaagacgtttgtgggtgattttcccaggagaagaaggtttagattatggaggtgtagcaagagaatggttctttcttttgtcacatgaagtgttgaacccaatgtattgcctgtttgaatatgcagggaaggataactactgcctgcagataaaccccgcttcttacatcaatccagaccacctgaaatactttcgttttattggcagatttattgccatggctctgtttcatgggaaattcatagataccggattttctttaccattctataaacgtatcctgaataaaccagttggacttaaggatttagaatctattgatccagaattttataattcgctcatctgggttaaagaaaacaacatcgaagaatgtggtttggaaatgtacttctcagttgataaagaaattctaggtgaaattaagagtcatgatttgaaaccgaatggtggcaatattcttgtgacagaagaaaacaaggaggaatacatcaggatggtagctgagtggaggttgtctcgaggtgttgaagaacagacacaagctttctttgagggctttaatgaaattcttcctcagcaatatttgcaatattttgatgcgaaggaattagaggttcttctgtgtgggatgcaggagattgatttgaatgactggcagagacatgccatctaccgccactacaccagaacaagcaagcagatcatgtggttttggcagtttgttaaagaaattgataatgagaagaggatgagacttctgcagtttgttactggaacctgccgattgccagtgggaggatttgctgaccttatggggagtaatggaccacagaagttctgcatcgaaaaagttggcaaagaaaattggttacccagaagccatacttgttttaaccgcctggacctcccaccttacaagagctatgagcaactgaaggaaaagctgttatttgccattgaagaaactgaaggatttggacaagagggatccggtaccgccaccatggtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgct

SEQ ID NO: 3 - DNA, 8775 bp, synthetic construct
  source 1..8775: mol_type = other DNA
cttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggtgatggagcagaagctgatttccgaggaggacctgaacggtggtagtctgaccatgaaatctcaacttcagatcactgtcatctcagccaaacttaaagaaaataaaaagaattggtttggaccaagtccttatgtagaagtcacagtagatggacagtcaaagaagacagaaaaatgcaataatacaaacagtcccaagtggaagcagcccctcacagttattgttacccctacgagtaaattatgttttcgtgtgtggagtcaccagaccctgaagtctgatgttttattgggaactgctggattagatatttatgaaacattaaagtcaaacaatatgaaacttgaagaagtagttatgactttgcagcttgtaggtgacaaagagccaacagagacgatgggagatttgtcagtttgtcttgatgggctgcaagtagaagctgaggttgttactaacggtgaaacgtcatgctccgagagtactacacagaatgatgatggctgcagaaccagagatgatacaagagtgagcacaaatggatcagaggaccctgaggttgcagcgtcaggggaaaacaagagggccaatgggaacaattctccgtccctttcaaatggtggttttaagccttctagacctcctagaccttcgagaccacctccacccaccccacgaagaccagcttctgtcaatggctcaccatccacgaattctgacagtgatggatctagtacaggctccttgccaccaacaaatacaaatgtaaatacaagtacatctgaaggagcaacatctggattaataattcctcttactatatctggaggctcgggccctaggcctctgaatactgtaagccaagctcccctaccacctgggtgggagcagagagtagaccagcatgggcgtgtttactatgtagaccatgttgaaaagcgaacaacatgggatagaccagaacctctacctcctggctgggaacggcgtgtggacaatatgggacgtatttattatgttgatcatttcacaagaaccacaacatggcagaggccaaccttggaatctgtccggaactatgaacagtggcagctacagcgtagtcagcttcagggagcaatgcagcagtttaaccagagattcatctatgggaatcaagatttgtttgctacatcacaaaacagagaatttgatccgcttggcccgttgccccctggatgggagaagagaactgatagcaatggcagagtgtattttgtcaaccacaacactcggattactcagtgggaagaccccagaagccaaggtcagttaaatgaaaagcccttaccagaaggctgggaaatgagattcacagtggatggaattccatattttgtggaccacaatagaagagcaactacttatatagatccacgaacgggaaaatcagccttagacaatgggccccagatagcctatgtgcgggacttcaaggcaaaagttcagtatttccggttctggtgccagcaactggccatgccacagcatataaagatcacagtgacaagaaaaacattgtttgaggattcctttcagcagatcatgagcttcagcccgcaagacctgagaagacgtttgtgggtgattttcccaggagaagaaggtttagattatggaggtgtagcaagagaatggttctttcttttgtcacatgaagtgttgaacccaatgtattgcctgtttgaatatgcagggaaggataactactgcctgcagataaaccccgcttcttacatcaatccagaccacctgaaatactttcgttttattggcagatttattgccatggctctgtttcatgggaaattcatagataccggattttctttaccattctataaacgtatcctgaataaaccagttggacttaaggatttagaatctattgatccagaattttataattcgctcatctgggttaaagaaaacaacatcgaagaatgtggtttggaaatgtacttctcagttgataaagaaattctaggtgaaattaagagtcatgatttgaaaccgaatggtggcaatattcttgtgacagaagaaaacaaggaggaatacatcaggatggtagctgagtggaggttgtctcgaggtgttgaagaacagacacaagctttctttgagggctttaatgaaattcttcctcagcaatatttgcaatattttgatgcgaaggaattagaggttcttctgtgtgggatgcaggagattgatttgaatgactggcagagacatgccatctaccgccactacaccagaacaagcaagcagatcatgtggttttggcagtttgttaaagaaattgataatgagaagaggatgagacttctgcagtttgttactggaacctgccgattgccagtgggaggatttgctgaccttatggggagtaatggaccacagaagttctgcatcgaaaaagttggcaaagaaaattggttacccagaagccatacttgttttaaccgcctggacctcccaccttacaagagctatgagcaactgaaggaaaagctgttatttgccattgaagaaactgaaggatttggacaagagggatccggtaccgccaccatggtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgct

SEQ ID NO: 4 - DNA, 7330 bp, synthetic construct
  source 1..7330: mol_type = other DNA
tcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccat

SEQ ID NO: 5 - DNA, 15419 bp, synthetic construct
  source 1..15419: mol_type = other DNA
tactacaaagacctgcgccgatgtcaaaacgaagcctggggcatggccgaccgcctgcgcattcagcaggccggacccaaggacatggtgcttctgtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatccccgggtactctagttaattaactagaggatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagac

SEQ ID NO: 6 - protein, 867 aa, Mus musculus
MEQKLISEEDLNGGSLTMKSQLQITVISAKLKENKKNWFGPSPYVEVTVDGQSKKTEKCNNTNSPKWKQPLTVIVTPTSKLCFRVWSHQTLKSDVLLGTAGLDIYETLKSNNMKLEEVVMTLQLVGDKEPTETMGDLSVCLDGLQVEAEVVTNGETSCSESTTQNDDGCRTRDDTRVSTNGSEDPEVAASGENKRANGNNSPSLSNGGFKPSRPPRPSRPPPPTPRRPASVNGSPSTNSDSDGSSTGSLPPTNTNVNTSTSEGATSGLIIPLTISGGSGPRPLNTVSQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQNKEFDPLGPLPPGWEKRTDSNGRVYFVNHNTRITQWEDPRSQGQLNEKPLPEGWEMRFTVDGIPYFVDHNRRATTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECGLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYTRTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQEG

SEQ ID NO: 7 - protein, 867 aa, synthetic construct
MEQKLISEEDLNGGSLTMKSQLQITVISAKLKENKKNWFGPSPYVEVTVDGQSKKTEKCNNTNSPKWKQPLTVIVTPTSKLCFRVWSHQTLKSDVLLGTAGLDIYETLKSNNMKLEEVVMTLQLVGDKEPTETMGDLSVCLDGLQVEAEVVTNGETSCSESTTQNDDGCRTRDDTRVSTNGSEDPEVAASGENKRANGNNSPSLSNGGFKPSRPPRPSRPPPPAPRRPASVNGAPSTNSDSDGSSTGSLPPTNTNVNTSTSEGATSGLIIPLTISGGSGPRPLNTVSQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQNKEFDPLGPLPPGWEKRTDSNGRVYFVNHNTRITQWEDPRSQGQLNEKPLPEGWEMRFTVDGIPYFVDHNRRATTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECGLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYTRTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQEG

SEQ ID NO: 8 - protein, 867 aa, synthetic construct
MEQKLISEEDLNGGSLTMKSQLQITVISAKLKENKKNWFGPSPYVEVTVDGQSKKTEKCNNTNSPKWKQPLTVIVTPTSKLCFRVWSHQTLKSDVLLGTAGLDIYETLKSNNMKLEEVVMTLQLVGDKEPTETMGDLSVCLDGLQVEAEVVTNGETSCSESTTQNDDGCRTRDDTRVSTNGSEDPEVAASGENKRANGNNSPSLSNGGFKPSRPPRPSRPPPPTPRRPASVNGSPSTNSDSDGSSTGSLPPTNTNVNTSTSEGATSGLIIPLTISGGSGPRPLNTVSQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQNREFDPLGPLPPGWEKRTDSNGRVYFVNHNTRITQWEDPRSQGQLNEKPLPEGWEMRFTVDGIPYFVDHNRRATTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECGLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYTRTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQEG

SEQ ID NO: 9 - protein, 862 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSESASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 10 - DNA, 2589 bp, Homo sapiens
  source 1..2589: mol_type = genomic DNA
atgagcgacagcggcagccagctgggcagcatgggcagcctgaccatgaagagccagctgcagatcaccgtgatcagcgccaagctgaaggagaacaagaagaactggttcggccccagcccctacgtggaggtgaccgtggacggccagagcaagaagaccgagaagtgcaacaacaccaacagccccaagtggaagcagcccctgaccgtgatcgtgacccccgtgagcaagctgcacttcagggtgtggagccaccagaccctgaagagcgacgtgctgctgggcaccgccgccctggacatctacgagaccctgaagagcaacaacatgaagctggaggaggtggtggtgaccctgcagctgggcggcgacaaggagcccaccgagaccatcggcgacctgagcatctgcctggacggcctgcagctggagagcgaggtggtgaccaacggcgagaccacctgcagcgagagcgccagccagaacgacgacggcagcaggagcaaggacgagaccagggtgagcaccaacggcagcgacgaccccgaggacgccggcgccggcgagaacaggagggtgagcggcaacaacagccccagcctgagcaacggcggcttcaagcccagcaggccccccaggcccagcaggccccccccccccacccccaggaggcccgccagcgtgaacggcagccccagcgccaccagcgagagcgacggcagcagcaccggcagcctgccccccaccaacaccaacaccaacaccagcgagggcgccaccagcggcctgatcatccccctgaccatcagcggcggcagcggccccaggcccctgaaccccgtgacccaggcccccctgccccccggctgggagcagagggtggaccagcacggcagggtgtactacgtggaccacgtggagaagaggaccacctgggacaggcccgagcccctgccccccggctgggagaggagggtggacaacatgggcaggatctactacgtggaccacttcaccaggaccaccacctggcagaggcccaccctggagagcgtgaggaactacgagcagtggcagctgcagaggagccagctgcagggcgccatgcagcagttcaaccagaggttcatctacggcaaccaggacctgttcgccaccagccagagcaaggagttcgaccccctgggccccctgccccccggctgggagaagaggaccgacagcaacggcagggtgtacttcgtgaaccacaacaccaggatcacccagtgggaggaccccaggagccagggccagctgaacgagaagcccctgcccgagggctgggagatgaggttcaccgtggacggcatcccctacttcgtggaccacaacaggaggaccaccacctacatcgaccccaggaccggcaagagcgccctggacaacggcccccagatcgcctacgtgagggacttcaaggccaaggtgcagtacttcaggttctggtgccagcagctggccatgccccagcacatcaagatcaccgtgaccaggaagaccctgttcgaggacagcttccagcagatcatgagcttcagcccccaggacctgaggaggaggctgtgggtgatcttccccggcgaggagggcctggactacggcggcgtggccagggagtggttcttcctgctgagccacgaggtgctgaaccccatgtactgcctgttcgagtacgccggcaaggacaactactgcctgcagatcaaccccgccagctacatcaaccccgaccacctgaagtacttcaggttcatcggcaggttcatcgccatggccctgttccacggcaagttcatcgacaccggcttcagcctgcccttctacaagaggatcctgaacaagcccgtgggcctgaaggacctggagagcatcgaccccgagttctacaacagcctgatctgggtgaaggagaacaacatcgaggagtgcgacctggagatgtacttcagcgtggacaaggagatcctgggcgagatcaagagccacgacctgaagcccaacggcggcaacatcctggtgaccgaggagaacaaggaggagtacatcaggatggtggccgagtggaggctgagcaggggcgtggaggagcagacccaggccttcttcgagggcttcaacgagatcctgccccagcagtacctgcagtacttcgacgccaaggagctggaggtgctgctgtgcggcatgcaggagatcgacctgaacgactggcagaggcacgccatctacaggcactacgccaggaccagcaagcagatcatgtggttctggcagttcgtgaaggagatcgacaacgagaagaggatgaggctgctgcagttcgtgaccggcacctgcaggctgcccgtgggcggcttcgccgacctgatgggcagcaacggcccccagaagttctgcatcgagaaggtgggcaaggagaactggctgcccaggagccacacctgcttcaacaggctggacctgcccccctacaagagctacgagcagctgaaggagaagctgctgttcgccatcgaggagaccgagggcttcggccaggagtaa

SEQ ID NO: 11 - protein, 862 aa, synthetic construct
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSESASQNDDGSRSKDETRVSTNGSDDPEDAGAGENRRVSGNNSPSLSNGGFKPSRPPRPSRPPPPAPRRPASVNGSPAATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 12 - DNA, 2589 bp, synthetic construct
  source 1..2589: mol_type = other DNA
atgagcgacagcggcagccagctgggcagcatgggcagcctgaccatgaagagccagctgcagatcaccgtgatcagcgccaagctgaaggagaacaagaagaactggttcggccccagcccctacgtggaggtgaccgtggacggccagagcaagaagaccgagaagtgcaacaacaccaacagccccaagtggaagcagcccctgaccgtgatcgtgacccccgtgagcaagctgcacttcagggtgtggagccaccagaccctgaagagcgacgtgctgctgggcaccgccgccctggacatctacgagaccctgaagagcaacaacatgaagctggaggaggtggtggtgaccctgcagctgggcggcgacaaggagcccaccgagaccatcggcgacctgagcatctgcctggacggcctgcagctggagagcgaggtggtgaccaacggcgagaccacctgcagcgagagcgccagccagaacgacgacggcagcaggagcaaggacgagaccagggtgagcaccaacggcagcgacgaccccgaggacgccggcgccggcgagaacaggagggtgagcggcaacaacagccccagcctgagcaacggcggcttcaagcccagcaggccccccaggcccagcaggccccccccccccgctcccaggaggcccgccagcgtgaacggcgcacccagcgccaccagcgagagcgacggcagcagcaccggcagcctgccccccaccaacaccaacaccaacaccagcgagggcgccaccagcggcctgatcatccccctgaccatcagcggcggcagcggccccaggcccctgaaccccgtgacccaggcccccctgccccccggctgggagcagagggtggaccagcacggcagggtgtactacgtggaccacgtggagaagaggaccacctgggacaggcccgagcccctgccccccggctgggagaggagggtggacaacatgggcaggatctactacgtggaccacttcaccaggaccaccacctggcagaggcccaccctggagagcgtgaggaactacgagcagtggcagctgcagaggagccagctgcagggcgccatgcagcagttcaaccagaggttcatctacggcaaccaggacctgttcgccaccagccagagcaaggagttcgaccccctgggccccctgccccccggctgggagaagaggaccgacagcaacggcagggtgtacttcgtgaaccacaacaccaggatcacccagtgggaggaccccaggagccagggccagctgaacgagaagcccctgcccgagggctgggagatgaggttcaccgtggacggcatcccctacttcgtggaccacaacaggaggaccaccacctacatcgaccccaggaccggcaagagcgccctggacaacggcccccagatcgcctacgtgagggacttcaaggccaaggtgcagtacttcaggttctggtgccagcagctggccatgccccagcacatcaagatcaccgtgaccaggaagaccctgttcgaggacagcttccagcagatcatgagcttcagcccccaggacctgaggaggaggctgtgggtgatcttccccggcgaggagggcctggactacggcggcgtggccagggagtggttcttcctgctgagccacgaggtgctgaaccccatgtactgcctgttcgagtacgccggcaaggacaactactgcctgcagatcaaccccgccagctacatcaaccccgaccacctgaagtacttcaggttcatcggcaggttcatcgccatggccctgttccacggcaagttcatcgacaccggcttcagcctgcccttctacaagaggatcctgaacaagcccgtgggcctgaaggacctggagagcatcgaccccgagttctacaacagcctgatctgggtgaaggagaacaacatcgaggagtgcgacctggagatgtacttcagcgtggacaaggagatcctgggcgagatcaagagccacgacctgaagcccaacggcggcaacatcctggtgaccgaggagaacaaggaggagtacatcaggatggtggccgagtggaggctgagcaggggcgtggaggagcagacccaggccttcttcgagggcttcaacgagatcctgccccagcagtacctgcagtacttcgacgccaaggagctggaggtgctgctgtgcggcatgcaggagatcgacctgaacgactggcagaggcacgccatctacaggcactacgccaggaccagcaagcagatcatgtggttctggcagttcgtgaaggagatcgacaacgagaagaggatgaggctgctgcagttcgtgaccggcacctgcaggctgcccgtgggcggcttcgccgacctgatgggcagcaacggcccccagaagttctgcatcgagaaggtgggcaaggagaactggctgcccaggagccacacctgcttcaacaggctggacctgcccccctacaagagctacgagcagctgaaggagaagctgctgttcgccatcgaggagaccgagggcttcggccaggagtaa

SEQ ID NO: 13 - protein, 862 aa, synthetic construct
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSESASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSREFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 14 - DNA, 2589 bp, synthetic construct
  source 1..2589: mol_type = other DNA
atgagcgacagcggcagccagctgggcagcatgggcagcctgaccatgaagagccagctgcagatcaccgtgatcagcgccaagctgaaggagaacaagaagaactggttcggccccagcccctacgtggaggtgaccgtggacggccagagcaagaagaccgagaagtgcaacaacaccaacagccccaagtggaagcagcccctgaccgtgatcgtgacccccgtgagcaagctgcacttcagggtgtggagccaccagaccctgaagagcgacgtgctgctgggcaccgccgccctggacatctacgagaccctgaagagcaacaacatgaagctggaggaggtggtggtgaccctgcagctgggcggcgacaaggagcccaccgagaccatcggcgacctgagcatctgcctggacggcctgcagctggagagcgaggtggtgaccaacggcgagaccacctgcagcgagagcgccagccagaacgacgacggcagcaggagcaaggacgagaccagggtgagcaccaacggcagcgacgaccccgaggacgccggcgccggcgagaacaggagggtgagcggcaacaacagccccagcctgagcaacggcggcttcaagcccagcaggccccccaggcccagcaggccccccccccccacccccaggaggcccgccagcgtgaacggcagccccagcgccaccagcgagagcgacggcagcagcaccggcagcctgccccccaccaacaccaacaccaacaccagcgagggcgccaccagcggcctgatcatccccctgaccatcagcggcggcagcggccccaggcccctgaaccccgtgacccaggcccccctgccccccggctgggagcagagggtggaccagcacggcagggtgtactacgtggaccacgtggagaagaggaccacctgggacaggcccgagcccctgccccccggctgggagaggagggtggacaacatgggcaggatctactacgtggaccacttcaccaggaccaccacctggcagaggcccaccctggagagcgtgaggaactacgagcagtggcagctgcagaggagccagctgcagggcgccatgcagcagttcaaccagaggttcatctacggcaaccaggacctgttcgccaccagccagagcagagagttcgaccccctgggccccctgccccccggctgggagaagaggaccgacagcaacggcagggtgtacttcgtgaaccacaacaccaggatcacccagtgggaggaccccaggagccagggccagctgaacgagaagcccctgcccgagggctgggagatgaggttcaccgtggacggcatcccctacttcgtggaccacaacaggaggaccaccacctacatcgaccccaggaccggcaagagcgccctggacaacggcccccagatcgcctacgtgagggacttcaaggccaaggtgcagtacttcaggttctggtgccagcagctggccatgccccagcacatcaagatcaccgtgaccaggaagaccctgttcgaggacagcttccagcagatcatgagcttcagcccccaggacctgaggaggaggctgtgggtgatcttccccggcgaggagggcctggactacggcggcgtggccagggagtggttcttcctgctgagccacgaggtgctgaaccccatgtactgcctgttcgagtacgccggcaaggacaactactgcctgcagatcaaccccgccagctacatcaaccccgaccacctgaagtacttcaggttcatcggcaggttcatcgccatggccctgttccacggcaagttcatcgacaccggcttcagcctgcccttctacaagaggatcctgaacaagcccgtgggcctgaaggacctggagagcatcgaccccgagttctacaacagcctgatctgggtgaaggagaacaacatcgaggagtgcgacctggagatgtacttcagcgtggacaaggagatcctgggcgagatcaagagccacgacctgaagcccaacggcggcaacatcctggtgaccgaggagaacaaggaggagtacatcaggatggtggccgagtggaggctgagcaggggcgtggaggagcagacccaggccttcttcgagggcttcaacgagatcctgccccagcagtacctgcagtacttcgacgccaaggagctggaggtgctgctgtgcggcatgcaggagatcgacctgaacgactggcagaggcacgccatctacaggcactacgccaggaccagcaagcagatcatgtggttctggcagttcgtgaaggagatcgacaacgagaagaggatgaggctgctgcagttcgtgaccggcacctgcaggctgcccgtgggcggcttcgccgacctgatgggcagcaacggcccccagaagttctgcatcgagaaggtgggcaaggagaactggctgcccaggagccacacctgcttcaacaggctggacctgcccccctacaagagctacgagcagctgaaggagaagctgctgttcgccatcgaggagaccgagggcttcggccaggagtaa

SEQ ID NO: 15 - DNA, 27 bp, synthetic construct
  source 1..27: mol_type = other DNA
ccacctccacctgctccaagaagacca

SEQ ID NO: 16 - DNA, 27 bp, synthetic construct
  source 1..27: mol_type = other DNA
tggtcttcttggagcaggtggaggtgg

SEQ ID NO: 17 - DNA, 33 bp, synthetic construct
  source 1..33: mol_type = other DNA
gcttctgtcaatggcgcaccatccacgaattct

SEQ ID NO: 18 - DNA, 33 bp, synthetic construct
  source 1..33: mol_type = other DNA
agaattcgtggatggtgcgccattgacagaagc

SEQ ID NO: 19 - DNA, 33 bp, synthetic construct
  source 1..33: mol_type = other DNA
gctacctcacagaacagagaatttgatccgctt

SEQ ID NO: 20 - DNA, 33 bp, synthetic construct
  source 1..33: mol_type = other DNA
aagcggatcaaattctctgttctgtgaggtagc